(19) United States
(12) Patent Application Publication
(10) Pub. No.: US 2013/0130294 A1
(43) Pub. Date: May 23, 2013

(54) NOVEL METHOD FOR CHARACTERIZING AND MULTI-DIMENSIONALLY REPRESENTING THE FOLDING PROCESS OF PROTEINS

(71) Applicant: Sigeng Han, Frankfurt am Main (DE)

(72) Inventor: Sigeng Han, Frankfurt am Main (DE)

(21) Appl. No.: 13/807,630

(22) PCT Filed: Dec. 28, 2010
(86) PCT No.: PCT/DE2010/001527

§ 371 Date: Jan. 11, 2013

(30) Foreign Application Priority Data
Jul. 5, 2010 (DE) 102010026094.0

(51) Int. Cl. C12Q 1/37

(52) U.S. Cl. CPC C12Q 1/37
USPC 435/23

(57) ABSTRACT

The invention relates to a novel method for characterizing and multi-dimensionally representing the folding process of proteins (FIG. 9). Said method comprises, in a methodically novel combination, kinetically arranging the hydrodynamic size of the refolding and thus modified protein, associating the proteolytically fragmented intermediates on the basis of the bioinformatic detection patterns, classifying the folding pathway association of the intermediates, characterizing the folding sequences, and multi-dimensionally visualizing the characterized folding process in a computer-aided manner. Said method is characterized in that all intermediates modified during the refolding and thus structurally blocked are identified and digitalized according to the four individual characteristics of said intermediates, namely the hydrodynamic size, the formation time, the folding pathway association, and amount. Said novel method has many applications in the field of research of protein folding and proteopathy, protein engineering, antibody engineering, molecular biology, therapeutic medicine, biotechnology, biotechnological production of protein pharmaceuticals, protein taxonomy, and nanotechnology for developing and producing novel functional protein materials. According to the invention, many and varied products in the form of different assay kits, devices, software, and machines can be produced and used to carry out said method.

### NOTATION

A new method for characterizing and multi-dimensional representing of the folding event of the proteins

### DESCRIPTION

The invention concerns a new method for characterizing and multi-dimensional representing of the folding event of the proteins. The subject of the invention is a several steps comprehensive method combined from the kinetic arrangement of hydrodynamic size of the refolded and thereby modified protein, the allocation (maping) of the isolated and proteolytically fragmented intermediate which is based on the bioinformation recognition model, the classification of the folding pathway of the dynamically modified intermediate, the elucidation of the folding processes and the multi-dimensional visualization of the characterized folding procedure.

In this new method the intermediates of the refolding and modified proteins are not just identified as by the conventional method along the pattern of their disulfide bonds but rather, according to invention, after their 4 individual characteristics namely hydrodynamic size, time of formation, folding pathway identity, and folding pathway population, identified and visualized multidimensionally. Thus not only the disulfidbond containing proteins but also the disulphide-free proteins can be examined, characterized along their elucidation of the folding processes without restriction of their molecular kind, their type and their size and visualized multidimensionally.

For the execution of this method, according to invention, various products can be manufactured and applied in the form of different assay kits, equipments, software and machines.

### THE FIELD OF THE INVENTION

The area of the invention covers the research of the protein folding and Proteopathy, the protein engineering, the anti-body engineering, molecular biology, the immunobiology, the therapeutic medicine, the biotechnology, the biotechnological production of protein medicines, protein taxonomy and the nanotechnology for the development of new functional protein materials.

### TECHNICAL BACKGROUND

Proteins have to be correctly folded, in order to be able to fullfill their biological function. The linear polypeptide chain after the synthesis at the ribosom has to be transferred in the appropriate secondary, tertiary and quaternary structure. Wrongly folded proteins are responsible for a number of diseases, which are at present in the discussion. To it belong numerous muscle diseases, but also Alzheimer's, the Creutzfeld Jakob disease, scrapie and BSE (Bovine spongiforme Enzephalopathie).

The development of an effective method for the clearing of the protein folding mechanism, for the explanation of the cause of the diseases and for the development of the new Protein therapeutic agents is until today a large challenge in the Life Science field. Since already Christian Anfinsen's (Anfinsen, 1972, Nobel price for chemistry) experiments for the refolding of proteins is well-known that the primary structure contains the entire information for the structure of a protein. However, thereby it is not explained how the proteins fold and find their native conformation. With the solution to a “problem of the protein folding” scientists intensively concerned world-wide in the last decades. Many theories and appropriate methods and techniques for examining, interpreting and characterizing the protein folding were recently developed.

### 1. Prediction of the Structure of Proteins

The structural prediction of the protein was used for the explanation of the folding mechanism. So far therefore are two well-known applicable methods known. First is the method which is based on the Alignment of the amino acid sequence, thus the homology modeling (Guex, 1997; Swede, 2003; Kopp, 2004) and the threading (Hendlich, 1990; Sippl, 1996; Madej, 1995). With these knowledge-supported methods a amino acid sequence from a unknown structure is examined by its compatibility with well-known protein structures. If a clear agreement is specified, the well-known structure can be consulted as output model. Until now this is the only practical and effective method, which is used for the tertiary structural prediction for homologous proteins. The second one is the ab-initio prediction (Karplus, 1990; Sippl, 1995; Shortle, 1997), in which one the folding of the amino acid chain should be predicted without further concerning of other well-known protein structures. With the help of computer-assisted computations it is tried to minimize the free enthalpy of a structure from a given amino acid sequence or to simulate the folding process. For this purpose, it is a world-wide structural prediction competition GASP (the Critical Assessment OF Techniques for protein Structure Prediction) (www.predictioncenter.org), 1994 created, which takes place every two years. In this competition protein sequences, whose 3D-Struktur stand shortly before the prediction, are published, the structural prediction were collected and afterwards with the experimental structures compared.

### 2. Classic Models for Protein Foding

In the classical aspect of the protein folding, which predominantly relies on experimental observations, there are a whole set of models. In the simplest case there is a two-state model. Most models assume however a folding in several steps. In all these models it is common, that they are correct for some proteins, for others in contrast not. Generally, in many cases secondary structure formation and the hydrophobe collapse are early folding events, in addition, the micro domain and puzzle (jigsaw puzzles model) folding model and/or the molten globule model (Ohgushi M & Wada A, 1983) have been realized in the folding of some proteins.

The two-state model is the simplest model for the folding of a small protein, in which there are only two stable states: folded or unfolded. The sequential model shows, that the folding from unfolded to folded goes over the intermediate. In the proposal of Tsong et al. a series of intermediates should define the path (Tsong et al., 1972). In the framework model (Kim and Baldwin, 1990) only single independent local elements of secondary structure are formed, which later diffuse to each other to form the tertiary structure. In contrast is the nucleation model (Wetlaufer, 1973) is based on the formation of a folding nucleus, from which the formation of native structures propagates. In the hydrophobic collapse model (Dill of al., 1995), the contraction of the polypeptide chain due to hydrophobic interactions of side chains is postulated as a first step, after one reorientation to the native structure at the local level.

In the micro-domain folding model first a small structural subdomains take their native conformation. These domains then grow through expansion or collision with other subdomains. In the puzzle-model exist like in all other models only one native conformation, but there are many different ways to achieve this end state, as well as a puzzle can be accomplished in several ways. The Molten globule hypothesis suggests that a Molten globule intermediate appears during the folding of all proteins. This folding intermediate has all the secondary structures of the fully folded protein, but no tertiary structure areas.

In all models at the beginning of the folding cascade the secondary structure elements (α-helices and β-sheets) are formed in the amazingly short time within the range of a nanosecond up to seconds. The further course of protein folding, is subjected to diffusion, collision, and contraction of the secondary structure and leads to the native structure of proteins, has a timescale of a few milliseconds to several days.

### 3. Thermodynamic Models for Protein Folding

From the experiments, that C. Anfinsen and others have done, it could be concluded that the native structure of proteins is a thermodynamically stable state and thus to the global minimum of Gibbs free energy available.

C. Levinthal (Levinthal, 1968) described that there must be a specific path (folding pathway) that is followed by a protein during its folding and under suitable conditions the protein pass through, a predetermined series of conformations (intermediate states) until it reaches the native state with the minimum energy state. In particular, models from statistical mechanics allows to replace this path concept of a sequential folding process by a funnel concept (Schultz, 2000) of parallel events.

The funnel concept of protein folding represents the free energy (vertical axis) of all possible protein structures as a function of the conformational degrees of variance (horizontal axis). Various stages of an unfolded protein on the upper side fall into the folding funnel. It has many local minima, in which protein can fall into. Some of these local minima represent intermediate stages (intermediate) on the path to the lowest energy of the protein native state. Some of this intermediates represents already a relatively stable compact structures “Molten Globules”, while others act as local minima trap and holds proteins in a misfolded state. The most protein molecules in accordance with this funnel model will not simply slip on their way to the folded state a smooth funnel, rather they will find probably more a rough and rugged landscape, in which they have to overcome all kinds of bumps, ditches, barriers and wells before they end up in a valley.

### 4. Kinetics of Protein Folding

Protein folding is a process that often consists of several phases

The fast phases of folding includes, for example, the hydrophobic collapse of the polypeptide chain, the formation of hydrogen bridges and the development of the secondary structure elements.

These go within the range of nano until microseconds, while slow phases run within the range of seconds until hours. Examples for a slow folding are RNase A (ribonuclease A) or thioredoxin, while for lysozyme and cytochrome c the fast phase dominates the folding process. However, there are only very few proteins that do not have at all slow folding phase.

It is known that large proteins usually fold more slowly and the folding of big proteins in the cell is supported by two different classes of additional proteins, which are called foldase and chaperones. Foldasen as PPI (peptidylprolyl c/s-trans isomerase in the periplasm), PDI and DsbA, B, C, D, G (protein disulfide isomerases in the periplasm) have a clearly defined catalytic activity, which accelerate the formation of covalent branches of the polypeptide chain. Chaperones, on the other side, perform many functions, at which function is probably the most important a creation of an environment for the nascent protein chain in which it can fold without causing the competing process of self-association. The bacterial chaperon GroEL (heat shock protein), for example, helps approximately one half of the medium sized (30-60 kDa), newly synthesized bacterial proteins in the folding. The distinction between foldase and chaperones can not always be clearly defined, since some proteins e.g. disulfide isomerase (PDI), is effective as foldase as well as a chaperone, in at least in vitro conditions.

### 5. Methods and Techniques for Studying Protein Folding

The experimental studies of protein folding can be range in two types. The first relates to experiments in the equilibrium state in which the conformations of the protein are shown as a function of the concentration of the denaturant or the temperature. The other relates to kinetic studies in which the structural changes of the protein are represented as a function of time at rapid changes in condition of the solvent. The present applied techniques, methods, and important chemicals for the studies of protein folding are:
NMR (nuclear magnetic resonance) spectroscopy, X-ray crystallography, electron microscopy and AFM (Atomic Force Microscopy) to study the dynamic structural change in the protein intermediate which is formed at un-und refolding and may be optionally separated by chromatography Spectroscopic methods such Fluorescence, UV (ultraviolet radiation)-NIS (visible spectrum) spectroscopy, ESR (electron spin resonance) spectroscopy, CD (circular dichroism) spectropolarimetry and Fourier transform infrared spectroscopy to study the structural changes of the protein in the unfolding und refolding, DLS (dynamic light scattering) and SLS (static light scattering) to measure the molecular radius of the proteins, ESI-MS (Electron spray ionisation-mass spectrometry) and MALDI-MS (matrix-assisted laser desorption/ionization mass spectrometry) in different types for mass determination of the protein and enzymatically cleaved protein fragments.
Stopped-flow, quench flow und gradient techniques fluctuation und jump methods, coupled with spectroscopic methods. These methods achieve a time resolution of a few milliseconds For a stop flow experiment, the refolding of a protein is triggered by rapid dilution of the denaturing solution or by drastic change in pH Using the so-called continuous flow techniques the folding processes can be examined up to the range of 50-100 ps.
Hydrogen-deuterium exchange in combination with spectroscopic methods for studying the kinetic folding pathway of the protein.
MD (molecular dynamics) for computer simulation of the molecular structure changes in the folding of the protein.
2D Paperelektrophorese native and, sodium dodecyl sulfate-polyacrylamide gel electrophoresis (SDS-PAGE), capillary electrophoresis (CE) and liquid chromatography, including the reversed-phase high performance liquid chromatography (RP-HPLC) for separation, analysis and verification of the isolated intermediates of the protein and the enzymatically cleaved protein fragments.
Fluorescence microscopy methods with spatial and temporal high resolution for the investigation of protein folding and protein transport within the cell, protein-protein interactions as well as structural changes of proteins during the folding: fluorescence depolarization, colocalize measurements, Förster resonance energy transfer (FRET), time-resolved resonance energy transfer (TRFREIT), fluorescence lifetime imaging (FLIM), fluorescence recovery after photobleaching (FRAP) and fluorescence fluctuation methods in different variants, in particular, fluorescence correlation spectroscopy (FCS).
Trapping and identification of disulfide intermediates. This happens, as the disulfide protein (oxidized protein) is reduced and unfolded by denaturation and reduction with known reductions und denaturing agents, and then released from reductions und denaturing agents, afterwards protein is subjected to reoxidation and during that modification of the thiol groups of cysteine residues with trapping reagents, for example iodoacetate, is performed and then intermediate products are trapped. These interstage products as intermediates are then separated by chromatography, proteolyzed, partially sequenced and optionally, depending on the position of the disulfide formation in protein fragments, identified by spectroscopy (Creighton, 1978).
The characterization of the folding process by assigning the single disulfide bonds in identified intermediates and schematic interpretation of their relationship (Creighton, 1988, Weissman & Kim, 1991).
Direct observation of the folding process of a single protein with atomic force microscope (Cecconi et al, 2005; Walther, et al, 2007).
The chemicals contain well known denaturing agents, reducing agents, reoxidant agents, modification agents, folding stabilizing agents, foldases, biochemical and chemical chaperones and inhibitors of folding and cell extracts etc.

### State of the Art of Characterizing the Process of Protein Folding

### 1. A Method for Characterizing the Process of Protein Folding

There were numerous pioneering work, in order to develop an effective method for characterizing the folding process of the proteins. A promising method, which is combined from the above described techniques and methods and offered in the form of a commercial product as a standardized method on the market for routine laboratory practice, for the characterization of the process of protein folding is so far not yet known.

That in the last decades world wide dominant conventional method for characterizing the folding process of the proteins involves mostly the small globular proteins and is limited to the disulfide-, also oxidative-known proteins and concerns very rarely large proteins with multi-domains. The method is based on the theoretical assumption that the order of formation of the disulfide bridges indexes the folding process of the protein. This method can be realized with a combination of two technical concepts, i.e. the identification and classification of the single disulfide containing intermediates of refolded protein and the subsequent interpretation of the folding pathways by correlation of the disulfide bonds order and potentially detected intermolecular rearrangements in the identified intermediates.

This arrangement of intermediates by the method based on their disulfide bonds was originally developed by TE Creighton (Creighton, 1978,1988). The protein is simultaneously in a one batch denaturated and reduced with denaturising agents GdmCl (guanidinium chloride) and reducing agent DTE (1,4-dithioerythritol) and by gel filtration from denaturising and reducing agent released. The reduced protein is then subjected to reoxidation to form a disulfide bond in a reoxidation buffer with GSH and GSSG (reduced and oxidized glutathione), wherein the chemical trapping agent iodoacetate or iodoacetamide is added at various time intervals to block the remaining free thiol groups and to stop the folding process. Thereby resulted trapped intermediates of refolding are afterwards with IEC (ion exchange chromatographie) isolated and further enzymatically cleaved into fragments and separated by two-dimensional paper electrophoresis. The identification of the intermediates is performed by determination of the single disulfide bond in the fragments by Edman sequencing (amino acid sequencing). The following characterization of the folding pathways takes place by allocation of the order of disulfide bond formation in the fragments.

This conventional method was improved by PS Kim and his colleague (Weissman & Kim, 1991) with a more rapid and sensitive method for the determination of disulfide bonds in the intermediates.

The starting material consists of the purified intermediates containing reoxidized disulfide bond and by the trapping agent iodoacetate blocked thiol groups. The disulfide bond of these intermediates is first reduced with a reducing agent to free thiol groups and then marked with a fluorescent iodoacetate derivative IAEDANS (5-[2-(2-lodacetamido)ethylamino]-naphthalen-1-sulfonic acid) to increase the detection sensitivity by the covalent bonds. The protein is then enzymatically fragmented. The labeled cysteine residues indicate the disulfide bond of the original fragments. The fragments are then with the increased separation resolution and speed separated by RP-HPLC (reversed-phase high performance liquid chromatography). By Edman sequencing and NMR analysis of the separated fragments, the positions of the disulfide bonds in the fragments are found. The intermediates are thereby identified depending upon the distribution of their single disulfide bonds. At the end the folding pathways can be schematically interpreted by the correlation of the disulfide bond order in the intermediates and analysis of intermolecular rearrangement. The folding process of a protein is so characterized.

This method pioneering by work of Creighton and Kim dominates since 20 years the practice of the characterization of protein folding worldwide. The folding studies of proteins represented in numerous publications are accomplished on the principle of this process and also in various modified forms depending on the preference of the art of technique composition. The investigations require large amounts of protein, sometimes even some 100 mg and usually it takes months, until the folding process of a small protein can be characterized by interpreting. The characterization results are often diversifying and can only be interpreted complementing among themselves.

According to the current specification of UniProtKB/TrEMBL nowadays are already 11 million proteins with characterized primary structures and more than 66,000 proteins with determined 3D structures (RCSB Protein Data Bank) for the necessary folding studies available, but until now, only a very small fraction of these proteins could be investigated by this method.

The folding pathways of three small proteins, RNase A (ribonuclease A), BPTI (bovine pancreatic trypsin inhibitor) and hirudin, as well-known model proteins were studied at best by this method, but only with limited accuracy and to some extent with a fundamental difference in opinion (Disulfide Folding Pathway of BPTI, Science vol. 256, 3 Apr. 1992). This proves that this method as the state of the art for the characterization of the folding process is not effective enough. Its implementation is complicated, it is working- and time consuming, it requires expensive equipment and has a low overall efficiency. Its results differ depending of the adopted technology used by the user. It could therefore not be developed into a standardized commercial product. The causes and the associated problems are in the unfavorable principle of this method and in the technical limitations which can be attributed to it.

### 2. Processing Problems of the State of the Art

The problem of the present method for the characterization of the folding process is that it is geared exclusively to the search for disulfide formation of protein intermediates and the separation and arrangement of the intermediates is generally concentrated only on the basic method of RP-HPLC (reversed phase high performance liquid chromatography) and CE (Capillary electrophoresis electrophoresis). This leads inevitably to the fundamental limitations of the method as follows:
First, about 30% of the total proteins that have no disulfide bond and which are not regarded as oxidative proteins are therefore excluded of this process, they can not therefore be studied.
Second, the disulfide formation as an indication for the folding in principle is suitable only for the study of small proteins, but not for the large proteins, because the separation and identification of the single disulfide intermediates are more difficult with the increasing protein size and the rising number of disulfide bond. A protein with 3 native disulfide bonds can generate 15 possible single disulfide containing intermediates, a protein with 4 native disulfide bonds has then 28 and a protein with 5 native disulfide bonds produces 45. This explosion like increase of intermediates and their molecular size encounter the separable limits of the HPLC method, which is suitable only for the separation of small proteins. Therefore, the method fails in principle for the larger proteins.
Third, many key intermediates in the disulfide-containing proteins whose thiol groups in the first phase of the refolding not form disulfide bridges, are not prosecuted as intermediates, and therefore artificially excluded from the study. The folding pathways can therefore be characterized and represented only incompletely.
Fourthly, the conformations of the single disulfide containing intermediates, which are grouped mostly gradually with decreasing molecular size, as exhibited by the present invention as an important proof of the origin of the folding pathways, are by the state of the art methods not differentiated, considered and recorded. This leads to a large loss of information about the details of the folding, in particular of the folding in the fast phase.
Fifth, the reoxidated protein, which is after the reduction exempt from denaturant agent and is kept for further use, is already due to the consequence of instinctive, spontaneous, and fast folding phase often in MoltenGlobule state, that has a similar molecular size as its slightly smaller native protein. This means that the following process, according to the characterization can only follow the last slow folding phase of Molten globule state to the renatured protein, but not the whole folding process.
Sixth, the RP-HPLC methodology can indeed separate the small intermediate fast, but it does not provide immediately relevant information about the temporal order of formation of various separated intermediates. This complicates and slows down enormously the process of identification of the intermediates and the characterization of the folding pathways.
Seventhly, the analyzed proteins are examined depending on the preference of the technique combination and on the modified versions of the conventional method and refer indeed more or less to all known new techniques and methods, for example, ERI-MS and MALDI-MS, etc. but because of the fundamental limitation of the method, their efficiency is ineffectively and therefore minimally exploited.
Eighth, the expensive Edman sequencing is used as an indispensable tool for the identification of the intermediates.
Ninth, the method of the state of the art can in most cases, as shown above, follow only a limited fraction of the folding process, but it is still not able to describe the course of this process in detail, because this method can not identify and characterize all occurring intermediates, just only the so-called significant intermediates.
Tenth, the results of investigations by the process of protein folding can not be accurately represented in a coordinate system, but only interpreted schematically.
Eleventh, the method is expensive, time consuming and is not able to standardize, to miniaturize and to automate

### OBJECT OF THE INVENTION

The object underlying the invention is to provide a novel method for effective and efficient characterization of the folding process, for both the disulfide containing and the disulfide free proteins. The new method should be able to study the protein folding without limitation of their kinds, types and sizes, to detect all occurring folding pathways and corresponding intra-molecular rearrangements, to identify the process of protein misfolding, to characterize the full event of both the slow and the fast phase of folding and to visualize descriptively multidimensional the characterization results and in various forms.

The new method can be used for its various applications in different molecular environments and embodiments with various physicochemical and biochemical conditions for elucidation of the mechanism of folding, misfolding, aggregation, the interaction, the self-assembly, the polymerization, aging, erosion and the nascent biosynthesis of proteins, for rationalization and increasing of the efficiency of the antibody engineering and protein engineering, for improvement of the activity and functionality of the proteins, for optimization of the biotechnological production of target proteins, for the development of nano-protein materials and for enrichment of the protein taxonomy etc.

In particular, the methods are used for the development of the biotechnological production process of in vivo simulated proteins, which are already known as post-translationally modified proteins for the search for novel biological and chemical agents and protein therapeutics through its influence on protein folding and degradation.

The new process should use less protein material, it should be easy to use, it should be time saving and also standardized, automatized and miniaturized. The instruments used for the execution of the invention in form of different assay kits, equipment and software, including the specific design and design for automation of the process, should be available. A transferable in a multidimensional energy landscape of folding multi-way model system for the optimal design and application of the method should be established.

### The Solution to the Problem

Above-mentioned object is achieved according to the characteristics of the patent claims of the present invention. The special feature of the solution lies in the fact that the intermediates of the refolding and thereby modified proteins are not just identified as by the conventional method along the pattern of their disulfide bonds but rather, according to invention, after their 4 individual characteristics namely hydrodynamic size, time of formation, the affiliation of the folding pathways, and amount. Based on that the characterization has a high total efficiency. In such manner characterized process of protein folding is no longer as in the prior state of the art interpreted schematically, rather it is represented with greater accuracy and in its diversity multidimensional visualised. The design, verification, optimization and rationalization of the process steps are supported by a new established multi folding pathway model. The method based on the invention is therefore clearly superior to the state of the art for the characterization of protein folding

### SUMMARY OF THE INVENTION

The invention combines a multistep method from the kinetic assembly of the hydrodynamic size of the refolding and thereby modified protein, of the bioinformatic recognition pattern based assignment of the separated and proteolytically fragmented intermediates, the classification of the folding pathways affiliation of the modified intermediates, the characterization of the folding processes and computer-aided visualization of the characterized folding process in a multi-coordinates system. This method refers to the designed multiple dynamic modifications of the refolding proteins, the modification and improvement of the key technology for the separation of the intermediates and established new methods for the identification of the folding pathways of the intermediate. The invention is based mainly on the composition created by the inventor of the following fundamental concepts and thereby new established multi-folding pathways model:

1) The process of refolding of a previously optimal fully unfolded protein can be indexed according to its gradually decreasing hydrodynamic size corresponding to its decreasing energy state, where at the changing structures of the folding protein by spatial disorder were made with chemical or enzymatic modification at various time intervals. They were stopped in their folding and braced, as intermediates separated and in order of their hydrodynamic size shown.

The hydrodynamic size is defined here as a predominantly with the hydrodynamic radius of a protein-described characteristics, which can be further enriched by continuous information about the structure, the distribution of charges, polarity, hydrophilicity, hydrophoby and on the molecular surface, etc. The hydrodynamic radius Rh, (or Stokes' radius) is the radius of the hydrodynamically equivalent spherical protein and is therefore unlike other well-known figures from the statistical analysis of polymers not static, but phenomenologically defined. According to the invention directly to the hydrodynamic radius shown hydrodynamic size describes the effect of the proteins in transport processes (viscosity, diffusion, permeation) and depends strongly on the shape and form of proteins. The intermediates of a protein, formed at specific time, which have individual conformations can, therefore, with their hydrodynamic sizes, that correspond to different energy states, be easily differentiate.

The hydrodynamic size may differ considerably from the real size of the particle and is usually smaller than the effective size of the particle. But this affects in no way the methodology, the hydrodynamic size as an index for the intermediates of the protein which should be tested is indicated, because here is the question primarily about the relativity of the hydrodynamic size and the resulting size order of the intermediate, and not about the absolute size.

These by modifying intercepted hydrodynamic sizes may differ from the actual hydrodynamic sizes at modification time. But these variations are limited to the structural variation between sequentially neighbouring intermediates and they can not change the order of the real hydrodynamic size of the intermediates and therefore do not harm the principle of the methodology. The reason for this is that during the refolding at different time intervals occurring modifications were carried out usually at the surface of the intermediates due to the gradual structural reduction and thereby more limited accessibility of the reagents. This may not lead to the development of highly energy-unfolding, but only to disturb the refolding and further trapping of the intermediates. Due to these disturbance caused structural changes are limited to such low energy differences that they are not strong enough to change the sequences of real hydrodynamic size and to overcome the underlying energy barriers. The hydrodynamic size can be spectroscopically (light scattering, fluorescence correlation, electron paramagnetic resonance, nuclear magnetic resonance spectroscopy and fluorescence polarization, etc.) preferably with DSL (dynamic light scattering) determined.

The hydrodynamic size is here also with radius of gyration (RMS-radius, root mean square radius) equivalent defined and can be spectroscopically measured preferably with SLS (static light scattering).

The hydrodynamic sizes of the intermediates are on principle proportional to their energy levels and can therefore if necessary be replaced with their spectrometrically determined thermodynamic parameters.

2) Intermediates of the protein which are produced during the refolding are varied and may, according to their common characteristics in the different groups be arranged according to the respective folding pathways. They do not only have different hydrodynamic sizes, but also a variety of structural designs, which decides on the accessibility and effectiveness of targeted modifications and serves as the basis for differentiation and identification of intermediates. This allow, those amino acid residues, e.g. lysine-, cysteine-, tyrosine-, histidine-, arginine-, tryptophan-r, methionine, glutamate- and aspartate residues of the folding protein in specific time intervals with specific side chain reagents due their structural accessibility dynamically and covalently to modify.

The resulting modified intermediates with the intercepted structures are in different hydrodynamic sizes and are based on both the varied placement of the modification as well as on the temporal decrease in the modification efficiency due to the structural reduction and the subsequent decrease of the accessibility of the reagents. The intermediates with common imprint are arranged in a particular group, which belongs to a specific folding pathway. As a result, the trapped intermediates differ not only on the hydrodynamic size and the time of formation, but also on its folding pathway.

These at different time intervals intercepted intermediates can either, according to the invention, with the modified and improved native polyacrylamide gel electrophoresis be separated, differentiated, over its size and amount, which is designed as a fourth characteristic of the intermediates, followed kinetically and with their hydrodynamic size as a function of folding time represented two-dimensionally, or first separated by liquid chromatography, quantified and then differentiated according to need and desire with the spectroscopic and/or thermodynamic process according to their hydrodynamic size, and then in two-dimensional form, as hydrodynamic size (possibly with a quantifiable amount) as a function of the folding time presented again.

This two-dimensional representation presents a refolding fingerprint profile of a protein, which is especially described by three characteristics of the intermediates, the hydrodynamic size, the time of their formation and the quantified amounts.

The folding pathways of all trapped and separated intermediates in the refolding can be classified by using the following criteria, which are also supported by the theory of evolution and the protein folding kinetic:
the Intermediate appearing first with the largest quantity belongs mostly to native dominant folding pathway and on this folding pathway appears least intermediates,
the Intermediate belonging to the native folding pathways has mostly a convergence structure and intermediate which does not belong to the native folding ways or to the missfolding pathway possesses however often DLS detectable divergence structures,
the Intermediate appearing in same quantified amount belongs mostly to the same folding pathway and this quantity decides on the width of the folding way or the diameter of the folding channel, thus the competency of a folding pathway.

The more exact classification of the folding pathway affiliation begins with proteolytic and chemical fragmentation of the isolated intermediates. The subsequent classification of the fragments is based on the bioinformatic comparison to the recognition of the theoretical fragment mass pattern. Here, the classification of the fragments is based on the distribution of their specific molecular weights, which are based on the types and extents of modification, and among each other possibly made binding patterns of disulfide formation and/or the inserted cross-linkers, to identify the folding pathways belonging grouped intermediates and to get the conclusion of the paths belonging to the folding.

Hereby mass spectrometries ESI-TOF-MS, electrospray ionization time of flight mass spectrometry and MALDI-TOF MS, matrix-assisted Laser-desorptions/-Ionisations-time of flight-mass spectrometry) are used to measure the number of the fragments and their molecule weights.

The resulting intermediate can be assigned according to their common characteristics in the respective groups. The over time grouped intermediate index in each case a folding pathway. The folding process of a protein is full characterized when all existing folding pathways are identified.

Hereby was found according to the invention:
An optimum fully to random coil unfolded protein contains at least a small fraction of the structurally slightly shaped primer populations. These populations can be regarded each as a mixture of the initial intermediates, which have different initial secondary structures in their very own micro-domains and which are distributed hierarchically and grouped with very low energy differences and thus indicating the origins of the folding pathways or sources of folding channels.
The refolding of a protein can be divided into four independent time scale and pace succession consecutively occurred phases: in nano to milliseconds super fast Phase I for the formation of the populations of the seed structures for the start of construction of the folding pathways, in micro to seconds fast Phase II for the construction of the parallel folding pathways, in the seconds to minutes phase III for the subsequent passage through the folds along the constructed path and, depending on structural composition of the respective protein in various tempos occurred Phase IV for the further intramolecular rearrangements of the micro regions and the evolution of the tertiary structure.
The populations of different seed-structures in Phase I are not incidentally formed, but by the primary structure of the protein defined. Further they decide on the following folding pathways. The parallel developed folding pathways in Phase II are various and from each other independent. Each folding pathway consists of several grouped metastable intermediates, which mark the stepping-stones of the respective folding pathways. The Phase II is completed at the time when the renatured protein occurs, and the highest number of intermediates appears. The refolding in phase-III runs parallel along independent folding pathways built in the Phase II and follows the same time emerging kinetics in Phase II to the time when all initially non-structured populations in the pool of unfolded protein and most intermediates in the folding pathways are no longer available. In phase IV the native structure of a renatured protein is completed.
Each resulting intermediate can be identified and described with at least 4 own individual characteristics, namely the hydrodynamic size, time of formation, the affiliation of the folding pathways, and amount.
The folding pathways may be classified into 3 types according to their general folding competence and each referred to as primary, secondary and misfolded way. These folding pathways may continue in parallel with its own subordinated folding pathways. The folding of the primary folding pathway dominates the folding process. The folding along the secondary folding pathway provide at the end appropriate intermediates with flexible among native-like structures that can convert in an intramolecular rearrangement to the native protein. The folding along misfolding pathways provides erroneous intermediates that convert either very slow in an intramolecular rearrangement to the native protein or left until the end of the fold as a misfolded protein.
The secondary and misfolding pathway can unite with each other through their branches or on the primary folding path way they can join each other along an energy low direction. The subordinated folding pathway can also merge with its main road by a branch, this creates a certain intermediate, which belongs to the two folding pathways.
The ratios of the parallel folding pathways can by regulating of the reaction conditions e.g. the concentration of the denaturation, reoxidation- and stabilizing agent, the pH and ionic strength, temperature, the use of chaperones or foldase etc. be changed. The refolding of a protein can thereby be accelerated e.g. in favor of the folding along the primary folding pathway in order to increase the productivity of a protein drug obtained by a renaturation, or to be controlled slowed down in order to reduce the protein aggregation or to facilitate the characterisation of the folding process. But the main folding ways defined by the primary structure of the protein and their subordinate folding ways thereby can not be changed respectively, no folding pathways will disappear thereby. Hereby intensified folding pathway thereby should not be identified as new folding way and the more weakly getting folding pathways thereby should not be ignored. There may also exist the intermediate, which is part of the misfolding path way, which has a hydrodynamic size and thus an energy state lower than its native protein.
The structural heterogeneity of a refolding protein decreases first up to the end of Phase 1 super fast and excursively and is then reduced on average more slowly and gradually until the end of Phase IV. The kinetic milestones of the decrease of structural heterogeneity are accordingly to the levels of the speed limit by the relatively stable structures of the intermediates visible and are located at the end of Phase-1 to -3. The decrease in structural heterogeneity thus appears gradually and is generally proportional to the decrease in hydrodynamic size of a refolding protein. The decreasing process of structural heterogeneity during the refolding of a protein is dependent on its nature, its type and thus on its folding pathways. The folding process of a protein can be mainly characterized by the entire characteristics of all intermediates under standard conditions occurring in the phases of folding-1 and -2 and are referred to as folding fingerprint of a protein.
Based on this new found knowledge to a funnel-energy landscapes corresponding multi-way folding model (FIG. 11) is established. The model is transferable in a multidimensional coordinate system. The 4 phase foldings are in this model further illustrated with 5 often in experiments appearing functional zones each in different graphical forms, wherein the energy state and the hydrodynamic size are plotted correspondingly as a function of folding time. This is described and interpreted below:
The highest zone-A acts as a pool of random coil in unfolded protein, with some slightly different structured populations. Due to the instinctual drive and the structural availability is the refolding of the Phase I in milliseconds super fast. Refolding in Zone B forms some dominant structural populations, which possess the initial native secondary structures and similar hydrodynamic sizes and energy levels. The refolded protein achieves its first speed limit due to the exhaustion of the creation of preferred secondary structure.
The construction of the multi-folding pathways in Phase II begins with the first occurring intermediate at the end of the zone B, and is ended with the appearance of the renatured protein in the zone-E. The populations of the initial native secondary structures, also known as seed population of the respective folding pathways, overcome first the individual main energy barriers and fold with its own speed over some structurally metastable levels, which appear in the form of intermediates and are referred to as marked stepping stones on the respective folding pathway, by the molten globule-state until to the renatured structure.
Along the primary folding pathway folding of the population including its potential subpopulations are simultaneously formed native secondary structures that are dominated the refolding. The refolding along this way with a very fast rate provides rarely intermediate. The secondary folding pathway comes from the population with partially formed native secondary structures and therefore consists of more or less native intermediates. The misfolded pathway arises from the populations with the faulty formed non-native secondary structures and is often accompanied by several non-native intermediates. The main folding pathway parallel to the accompanying subordinate folding pathways originate from structural subpopulations, with secondary structure formation respectively deviating and lead usually during the folding by the intramolecular rearrangement back to the main folding pathway.
The potential complexity of the multi-folding pathways in this model is represented symbolically It is shown that any form of seed population, which each of them is responsible for the formation of the symbolized primary, secondary and misfolding pathways, initially should overcome 3 different intensity defined energy barriers in order to achieve access to the folding pathways, and then in each case along the 3 alternative folding pathways refold with individual side-energy-barriers in which corresponding intermediates are developed. Thereby may occur at least 27 folding pathways, without taking into account the provided branched folding pathways. But the refolding of a protein can usually be done depending on its own nature only very limited on certain related parallel folding pathways.
The folding pathways get together at the Molten globule state in Zone-D. The most of intramolecular rearrangements, including the native-oriented disulfide rearrangements of the intermediates take place from different folding pathways.
Along the multi-folding pathways and the Molten-globule state refolded and first in Zone E renatured protein has the varying and flexible structural regions, which are based on the protein evolution and is responsible for the retention of its adaptability and functionality with respect to the substrates. This renatured protein rearranges in its native structure continuously completely during the continued flow of the folding in Phase III and until the end of Phase IV. The zone-E represents not only the renaturated and native structure of the protein, but also through intramolecular rearrangement structural variants, including the misfolded structures whose hydrodynamic size and energy state are sometimes even lower than natively.
The model shows four speed limits of protein folding. The first is at the end of Phase I and also at the same time at the main entrance of the energy barriers of the folding pathways. The second are the gradual occurring side-energy barriers, appearing in the form of sequential intermediates on the folding pathways. The third occurs when Molten globule state as a result of intramolecular rearrangement barriers and collapse barriers to renaturated protein. The fourth goes back to the rearrangement barriers to complete the native structure of the protein. The first and third speed limits have a strong bottleneck effect. They decide mainly on the folding rate of a protein. The different strengths of the speed limits are quantitatively determined by analyzing the amount of each distribution of the accumulated intermediate on the limited time stages
The model means that the folding process of a protein can be characterized by the identification of parallel in the speed limit stages and in between resulting intermediates. A muster consisting of the folding pathways and involved intermediates can be provided thereby as the basis of the experimental fingerprint of folding.
The model shows, that the folding on multi-folding pathways in principle is not cooperative. The cooperative behavior will happen only if the folding is dominant in the primary folding pathway.
The model is applicable to all proteins. Each protein, whether folded on the Single folding pathway or multi folding pathway, super fast or slow or first super fast and then folded slowly, or only slowly folded, can be found in this model according to its own folding pathways. The model is therefore among others used especially for improving the design, rationalization of the review and orientation of the optimization of process steps and thus for the optimal design and application of the inventive method.

3) Thereby characterized folding process can be represented multidimensionally first by the digitalization of characteristics of all intermediates and the hydrodynamic size of all the intermediates in each case against its time of formation, the folding pathways and possibly their quantified amounts in a multi-dimensional coordinate system. The representation of the folding process can be visualized in diversity through new combinations of coordinates defined on these four characteristics and the use of computer graphics. Hereby it is possible easily to determine for example the folding pathways (the construction of fold channels), their kinetic process, their percentage contribution to the folding, the process of misfolding, the formation of intermolecular rearrangement, the order of disulfide formation, the effects of biological and chemical factors influencing folding, the process of start of the aggregation of the proteins, the in vitro effects of a folding inhibitor or chaperone on the activity, function and the degradation of a protein as well as the procedure of a simulated in vitro dynamic co- and posttranslational modification illustrated etc. from the graphic representation of a characterized folding process. The relationship between three-dimensional structure and energy landscape of all intermediates of a refolding protein, can be presented according to the funnel concept (Schultz, 2000) qualitatively and quantitatively and also visualized multidimensionally in other various forms of energy landscape.

### DETAILED DESCRIPTION OF INVENTION

The process according to the invention will be presented firstly by simple description of respective steps and secondly by a detailed description.

### 1. General Analytics of the Characterized Protein

Based on the analysis of the primary, secondary and if applicable three-dimensional structure and biological and physicochemical properties of the protein, mass spectra fragmentation are predicted and then it is decided which approach should be first applied; e.g. which reagent for which amino acid specific modification should be used or which method should be used for separation of intermediates.

2. Separation of Optimal Unfolded Protein Sample with Maximum Hydrodynamic Size.

Protein is denatured, reduced and when necessary reducing reagents are removed. The chromatographically separated protein sample has the maximum hydrodynamic size while disulfide bridges of the protein containing disulfide bonds are completely reduced to free thiol groups. Thus protein is completely denatured.

### 3. Dynamic Protein Modification

The refolding protein is modified after various time intervals during refolding with or without influencing factors with reagents reacting with amino acid side chains according to their accessibility. The degree and pattern of modification is depended on individual characteristics of the intermediate with its diverse and relative stable conformations; namely hydrodynamic size, time of formation, folding pathway identity and amount. Those characteristics are used to identify each intermediate and enable separation.

### 4. Separation and Quantification of Intermediates and Two-Dimensional Presentation of Hydrodynamic Size and Amount as Function of Time of Formation.

Intermediates that are trapped after various time intervals during refolding by modification are preferred separated by improved gel electrophoresis according to the invention and are quantified by scanning intensity of gel bands. Different gel bands contain intermediates with different hydrodynamic size as function of refolding process which is two-dimensional presented on gel. They (intermediates) first can be separated by liquid chromatography and then can be analyzed by spectroscopy in order to differentiate by hydrodynamic size and in order to quantify the amount. Subsequent analysis allows a multi-dimensional presentation of 4-phase multi-pathway folding model while the hydrodynamic size and amount is a function of time during refolding. All intermediates are tabulated according to hydrodynamic size, amount and time of formation. The hydrodynamic size of intermediates can be exchanged by any other thermodynamic parameter after their individual separation.

The fingerprint of protein refolding with all intermediates that form until the end of refolding and which differentiate by hydrodynamic size, amount and time of formation can be generated either by two-dimensional gel electrophoresis or by digital (graphical) presentation of analysis after separation and quantification with liquid chromatography.

5. Fragmentation of Intermediates with in-Gel-Digestion or in Solution Digestion.

With the fragmentation it is aimed to differentiate folding pathway identity more precisely. All intermediates that are separated by hydrodynamic size by gel electrophoresis or by micro-chromatography are preferred first digested by trypsin. The fragmentation can be performed with other endoproteases Lys-C, Glu-C and Asp-N if this is necessary or with other exoproteases or with chemical probing.

### 6. Mass Spectroscopic Detection of Fragmented Intermediates

The molecular weight of all fragments including fragments that are bigger in size because they are connected with disulfid bonds and/or cross-linker are detected with mass spectroscopy (ESI-TOF-MS, Elektrospray Ionisation-Time-Of-Flight-mass spectroscopy) and MALDI-TOF-MS (Matrix-assisted laser desorption/ionisation) and then are tabulated in a data bank. For more precise differentiation of similar fragments MALDI-TOF-MS/MS (Tandem MS) or MALDI-TOF-MS-PSD (Post-Source-decay), or additional exoenzymatic and/or chemical hydrolysis can be used.

### 7. Analysis of Folding Pathway Identity of Intermediates

Comparison of experimental mass spectroscopy fragment pattern and predicted theoretically possible pattern allows analysis of the type, number and location of modification which presents individual characteristics for each intermediate. By identifying these characteristics intermediates can be grouped according to their similarity to other intermediates and they can be assigned to specific folding pathway according to their group membership. Once intermediates are assigned to a group membership they are also assigned to specific folding pathway identity. Such analysis is compensated and complemented by criteria that are supported by previously described theory of protein evolution and protein folding kinetics.

### 8. Identification and Classification of Intermediates

Each intermediate can be assigned by measured hydrodynamic size, time of formation, quantified amount and folding pathway identity and each intermediate can be defined by those 4 characteristics. All identified intermediates can be separated into groups according their common features and further classified into different folding pathways. Intermediates that belong to the same folding pathway are characterized by the presence of same modification pattern, gradually decrease in hydrodynamic size and the time of formation of each intermediate of the same group.

### 9. Characterization of the Folding Process

With the parallel presentation of all intermediates belonging to different folding pathways with their identified characteristics in a multidimensional coordinate system in a multidimensional coordinate system it is possible to present the dominating pathway of native folding, pathway of fast and slow folding, pathway of misfolding, folding kinetics in all pathways, sequence of disulfide bond formation and/or cross linker formation, the intermolecular rearrangement and the folding pathway branches, enlargement, crossing, traversing and conjunctions and all involving intermediates are directly determined.

The protein folding pathway is than characterized.

### 10. Graphical and Visual Presentation of Protein Folding Process

With computer graphics the characterized folding process can be visualized in a multidimensional coordinate system in different presentation types or can be used for animation while folding pathways also can be presented as folding channels for folding funnel.

Realization of the method can happen step by step manually and/or completely automated while means for completion are different assay-kits, devices and software as well as specially developed and designed machines.

### 1. General Analytics of the Characterized Protein

First step is the analysis of the primary, secondary and tertiary structure and analysis of the physicochemical properties of the characterized protein. Based on that it is decided which procedure is appropriate for the characterization. Chosen procedure and concept include description of all steps with all individual handlings, modification types, side chain specific reagents and method for separation of intermediates and their differentiation according to their hydrodynamic size including reaction conditions e.g. protein concentration, solvent, ionic strength, pH and temperature.

According to the invention specifically developed program can be used for classification of the protein according to the molecular size, content of disulfide bonds, type of hydrophilic or hydrophobic consistence, single or multiple domains, for choice of side chain specific modification reagents, for synthesis and analysis of theoretical mass spectroscopy fragment pattern based on proteolytic digestion of modified protein intermediates and for presentation, differentiating and analysis of theoretical mass spectroscopy fragment pattern in correlation with all types of modification according to the invention.

For the analysis related to the application of invention it is possible to use additional programs e.g. for presentation and analysis of the theoretical mass spectroscopy fragment pattern of the in vitro post translational modification and proteolytic cleaved intermediates and for presentation and analysis of mass spectroscopy fragment pattern of the in vitro simulated refolding under chemical and/or biological factor of the modified and proteolytic cleaved intermediates.

2. Separation of Optimal Unfolded Protein Sample with Maximum Hydrodynamic Size.

The optimal unfolded protein has maximum hydrodynamic size. Separation of this protein sample is carried out after denaturing and reduction of the protein and is applicable to proteins with and without disulfide bonds. Protein with disulfide bonds is first denatured with denaturing and reducing reagents. Afterwards reducing reagents are removed chromatographically while at the same time protein is separated in different samples which are differentiated according to their hydrodynamic size. Removal of reducing reagents for specific proteins which have fast intermolecular reoxidation potential should happen gradually. Through spectroscopic analysis with e.g. DLS and determination of reduced thiol-residues in molecule with known Ellmann-reagents it is possible to determine whether analyzed protein is completely denatured and whether its disulfide bonds are completely reduced. The optimal separated protein sample with certainly fully reduced and denatured protein has maximum hydrodynamic size and is used as starting material for following dynamic modification.

Denaturing and reduction are conducted in same buffer solution with known denaturing and reducing reagents, e.g. Guanidinium chloride and DTE (1,4-Dithioerythrit). Concentration is in each case up to 20 mg/ml for proteins, up to 8M denaturing reagents and up to 0.4M reducing reagents. Optimal unfolding of the protein can be improved by increasing temperature, changing pH value and addition of other reagents. Chromatography used for removal of reducing reagents is preferably performed with buffer containing highly concentrated denaturing reagents with low pH value. Denaturing and/or unfolding of proteins without disulfide bonds is performed without reducing reagents. Control and determination of the protein sample with maximum hydrodynamic size is performed with spectroscopic methods preferably with DLS after liquid chromatographically separation.

### 3. Dynamic Protein Modification

Completely denatured protein has maximum hydrodynamic size. Through fast change in concentration of denaturing reagents after dilution, or through change in temperature or pH denatured protein will start to refold while hydrodynamic size is decreasing gradually. Protein folding is trapped in time-course manner with chemical or biological modification through sterical blocking of side chains. Protein is converted to versatile and structurally relatively stable intermediates while structure of intermediates is dependent on either used reoxidation reagents for the reduced proteins with disulfide bonds or cross-linker reagents for proteins without disulfide bonds with individual characteristics of newly formed disulfide bonds and cross-linker bonds. According to the invention it was found that diversity of modification pattern of proteins is fundament and essential requirement for complete characterization of the protein folding process. Furthermore according to the invention it was found that precision and accuracy of the characterization of the protein folding process especially of the fast folding phase is dependent on reagents and reaction rate of method. In this conception a multiplicity of possibilities for modification of refolding protein assures that all intermediates that are present during refolding can be modified with appropriate reagents and can be differentiated according to their structural characteristics and can be identified according their individual characteristics.

Modification can be performed in different ways depending on the aimed characterization and depending on way, type and size of characterized protein with appropriate approach and method as well as chosen chemicals and material which are combined for use. Protein modification is carried out according to the invention specifically at residues, e.g. of cysteine, lysine, tyrosin, histidin, arginine, tryptophane, methionin, glutamic acid, asparagine and aspartic acid including N- and C-terminus of refolding protein. Protein modification can be classified according to type, extent as well as purpose of application into different groups
dynamic modification of proteins with disulfide bonds
dynamic modification of proteins without disulfide bonds
dynamic modification of multi domain proteins
simulated dynamic in vitro co- and post translational modification
dynamic modification during simulated in vitro protein folding
dynamic modification during in vitro protein biosyntehsis

According to invention dynamic modification of proteins with disulfide bonds means that reduced and denatured protein with disulfide bonds is reoxidized and after various time intervals during reoxidation sample portions are isolated and modification is carried out, preferably performing single modification with side chain specific reagents that block free thiol groups of protein. According to invention dynamic modifications of refolding structures leads to trapping of various intermediates with different structural characteristics and hydrodynamic size. During this various native and non-native disulfide bonds which lead to specific pattern of interconnecting of proteolytically cleaved fragments are generated while remaining thiol groups are blocked through reaction with side specific reagents. Those disulfide bonds that are formed during refolding are basis for classification of intermediates into folding pathway identity. For exact differentiation of folding pathways and corresponding intermediates multi-manner modification should be applied while residues of cysteine, histidine, lysine, methionin and arginine should react with side specific reagents separably or exclusively with one single reagent e.g. Iodine acetamide under controlled reaction conditions, for example with stepwise variation of pH value.

According to the invention dynamic modification of proteins without disulfide bonds means that denatured proteins which do not have thiol groups to form disulfide bonds are refolded and sample portions are isolated after various time intervals and protein is than modified according to the characteristics of the sequence and structural accessibility of modified side chains and their enzymatic cleavage pattern of single modification, of multi modification and/or internal cross-linker modification. Single modification is addressed to individual amino acids which are frequently present in protein sequence and which can be modified with specific single side chain modification reagents. Multiple modification is suitable for amino acids which not individually but together are abundant in sequence and can be easily modified with at least one single side chain modification reagents. Those selected amino acids are than modified either by changing reaction conditions or by mixing parallel with different multiple specific side chain reagents. Internal cross-linker modification leads to formation of disulfide bridge-like bonds and therefore proteolytic fragments become connected. Such modifications add individual characteristics to the intermediate through individual type, number and site of inserted modification reagent to the side chain as well as individual mass spectroscopy pattern of proteolytically produced fragments, which are dependent on micro environment of refolding protein. Interdependence of individual modification pattern and refolding protein structure is fundamental for differentiation of intermediates according to the hydrodynamic size and for classification of intermediates without disulfide bonds.

According to the invention dynamic modification of multi domain proteins means that independent, isolated protein domains of multi domain protein or intra molecular dependent domains of inherent protein are after denaturation refolded and sample portions are separated after various time intervals. Subsequently selective single or multiple modification and/or cross-linker modification is carried out according to individual characteristics of the protein, its sequential, structural and proteolytic characteristic. For this purpose it is possible to use mono and multi functional reagents as well as reagents with biotinylation or other reagents in order to insert cross-linker between later proteolytically produced fragments and especially it is possible to use reagents for optimal denaturation of large multi domain proteins.

For multi domain proteins containing disulfide bonds it is necessary to completely reduce, denature and afterwards to remove reducing reagents before starting refolding and modification.

According to the invention simulated dynamic in vitro co and post translational modification is defined as
First, completely denatured protein without disulfide bonds and completely reduced and denatured protein with disulfide bonds freed of reducing agents are characterized according to the invention. Than protein refolding is carried out with in vitro post-translational modification with either chemical or enzymatic reagents or with cell extract that is specific for in vitro post-translational modification. During refolding and in vitro post-translational modification protein sample portions are separated after various time intervals. Intermediates are than separated with gel electrophoresis or chromatographic methods. In further steps folding process is characterized according to the invention and compared with and without post-translational modification.
Second, characterization of the post translational modification is carried out through addition of 15N and/or 13C isotope labeled protein and further quantification by analysis with mass spectroscopy. For this purpose protein with and without isotope labeling are mixed together in defined ratio and then characterization is carried out according to the invention.
Third, protein that has been characterized according to the invention is analyzed during in vitro biosynthesis in reaction with cell extract that is specific for biosynthesis and if present co-translational modification of protein. Analysis is carried out to characterize the process and extent of co-translational modification of the protein and to differentiate co- and post-translational modification by comparison of in vitro biosynthesized and if possible co-translational modified protein that is separated with gel electrophoresis and/or chromatographic methods, denatured and reduced, reduced reagents are removed, than refolding is carried out and if necessary reoxidation is introduced and in next steps according to the invention characterization is carried out and folding process analyzed according to the invention is compared with and without post-translational modification.
Fourthly, protein that has been characterized according to the invention for its simulated in vitro co- and/or post-translational modification is analyzed with same cell-free reaction with different candidate reagents that are responsible for regulation of physiological and biochemical conditions in order to scan for chemical and biological additives and inhibitors for such modifications and in order to analyze their influence on protein folding. In order to analyze the effect of such additives and inhibitors analysis is carried out and compared.
All thereby introduced in vitro simulated post-translational modifications can be coupled with the technology of protein immobilization and protein chip development that is incorporated in high-through-put-screening and according to the invention it can be used for further applications and depending on the aims it can be combined and expanded in different ways, e.g. for optimization of synthesis of in vitro post-translationally modified biologics.

Dynamic modification of simulated in vitro protein folding are defined according to the invention as following:
In first embodiment it is meant that completely denatured protein without disulfide bonds or a completely reduced and denatured protein freed from reducing reagents whose protein folding process has been characterized before according to the invention is analyzed in parallel reactions with different molecular environmental conditions with changes of protein concentration, temperature (including freezing and unfreezing), solvent, ion strength, pH value and additional reagents for stabilization and/or destabilization of the protein in order to characterize aggregation of protein during refolding to a simulated in vitro folding process. Reaction without aggregation is used as negative control. For this purpose protein is refolded and sample portions are separated after various time intervals. Afterwards selective modification with specific side chain reagents is carried out and sample is ready for other applications for characterization.
In a second embodiment it is meant that two or more completely denatured, reduced protein freed from reducing reagents that has been characterized according to the invention are analyzed in parallel reactions with different molecular environment conditions by changing protein concentration, temperature (including freezing and unfreezing), solvent, ion strength, pH value and other reagents for stabilization and/or destabilization of protein in order to characterize interactions of particular proteins during refolding. Reaction without interactions is used as negative control. For this purpose refolding proteins are analyzed separately in individual experiments or in one experiment while after various time intervals sample portions are separated than mixed and in each case selected modification with according side chain reagents is carried out, subsequently samples are ready for further characterization of the process.
In third embodiment it is meant that a completely denatured protein without disulfide bonds and a completely denatured, reduced protein with disulfide bonds freed from reducing reagents that has been characterized before according to the invention is analyzed in parallel reactions with different biological and/or chemical inhibitors or additives in order to search for biological and chemical reagents that have effect on protein folding for simulated in vitro folding process. Experiment without inhibitors or additives are used as negative control. For this purpose refolding protein is separated in portions after various time intervals and selective modification with according side chain reagents is carried out and subsequent separation and proteolytic cleavage and mass spectroscopic measurements are carried out in order to have characterization and comparison with other protein folding process.
In a fourth embodiment it is meant that a completely denatured protein without disulfide bonds or a completely denatured, reduced protein with disulfide bonds freed of reducing reagents that is characterized according to the invention before, is analyzed in parallel experiments with different known foldases or/and chaperone in order to analyze effect of foldases and cheperones on protein folding during simulated in vitro protein folding process. Experiment without foldases and chaperones is used as negative control. For this purpose refolding protein is separated in sample portions after various time intervals and selected modification with according side chain reagents, subsequent separation and further proteolytic cleavage and mass spectroscopic measurements are carried out in order to characterize and compare protein folding process.
In a fifth embodiment is meant that completely denatured protein without disulfide bonds or completely denatured, reduced protein with disulfide bonds and free of reducing reagents that has been characterized according to the invention with regard to the effect of foldases or/and chaperones is further analyzed in order to search for effective inhibitors of foldases and chaperones and their candidate inhibitors are analyzed during simulated refolding. Different candidate reagents can be analyzed in a parallel way while one experiment without candidate reagents is used as negative control. For this purpose protein is separated in portions after various time intervals and selective modification with according side chain reagents is carried out. After subsequent characterization and comparison of all protein folding process ability of candidate reagents to inhibit foldases and/or chaparones can be analyzed. According to the invention foldases and chaperones used for those experiments can be added directly to the reaction or through application of cell extract for cell-free protein synthesis before starting protein folding. According to the invention application of dynamic modifications during simulated in vitro protein folding process can be used to search for biological and chemical additives and inhibitors of protein folding and protein degradation for development of new biologics.
In a sixth embodiment it is meant that completely denatured polypeptide chains or proteins that have been characterized according to the invention are analyzed in parallel reactions with biological and/or chemical reagents that influence folding and if possible reagents that influence formation of peptide bonds under controlled conditions in order to analyze defined self assembly and polymerization of polypeptides or proteins during their refolding in order to develop and synthesize nano-protein material. Experiment without reagents that influence formation of peptide bonds is used as negative control. For this purpose refolding and at the same time self-assembly and/or polymerizing protein is separated in portions after various time intervals and selective modification with according side chain reagents is carried out and portions are ready for further characterization of folding process.
In a seventh embodiment it is shown that due to incorrect folding of disease-causing proteins whose folding process has been previously characterized according to the invention, in the parallel approaches with the biological or/and chemical candidate folding stabilizing substances which specifically bind to unfolded proteins and thus stabilize the protein structure and improve the folding or masking the hydrophobic domains of misfolded proteins, thereby increasing their solubility and prevent aggregation of unfolded proteins in the search for pharmacological chaperones to Proteopathy to a simulated in vitro folding process can be accommodated. The approach with no biological or/and chemical substances stabilizing folding candidate serves as negative control. Here, the refolding proteins which are subjected to folding stabilizing factors are separated in portions after various time intervals and are subjected to selected modification with appropriate side chain reagents, subsequent provided for the inventive characterization and evaluation of this process.
In an eighth embodiment it is shown that a specific prion protein whose folding process has been previously characterized according to the invention, in parallel reactions with the biological or/and chemical folding influencing substances under regulation of the destabilizing conditions such as temperature, pH and ionic strength for the characterization of the process for first refolding of PrP_{c} (Cellular prion protein=cellular prion protein) to PrPS^{c} (scrapie prion protein; pathogenic form of prion protein) including present aggregation and secondly the reversal of the PrPS to PrPc is brought, including the reduction of aggregation in order to clarify the pathogenesis and the search for treatment options and ways to prevent a simulated in vitro folding process. The approach with no biological or/and chemical substances influencing protein folding is used here as a negative control. In this case the refolding prion-protein subjected to folding influencing substances are separated in portions after various time intervals and a selected modification with appropriate side chain reagents is carried out and provided for further approach.
In a ninth embodiment it is shown that a completely denatured, reduced and free of reducing agent protein whose folding process has been previously characterized according to the invention, in its refolding either a catalytically accelerated isomerization, deamidation and racemization by supplying the photochemical and thermal energy, or one of radical reaction, oxidative stress and environmental influences initiated modification is subjected while refolding protein is separated in the portions after various time intervals and is then subjected to the modification with the same side chain reagents. The mixture without isomerization, deamidation and racemization or free radical exposure, oxidative stress and environmental factors serves as a negative control. Upon further characterization and comparison of the processes of nding events changes in charge conditions and conformation and protein aging are examined. This is the study of the aging process, the discovery and development of biological and chemical antioxidants.

Under the dynamic modification in the in vitro protein biosynthesis is to be understood according to the invention that in a cell-free approach biosynthesized protein whose folding process is according to the invention characterized before, during its rapid biosynthesis is separated in portions after various time intervals with cooling preservation, then mixed together, denatured, optionally reduced and freed from the reducing agent and according to the defined length of the amino acid chains separated selectively in different polypeptide fragments by gel electrophoresis or chromatography, wherein each of these fragments has a certain length of the amino acid sequence may have equal molecular masses, but unequal structural design and is referred to as a mini-intermediates. These separated polypeptide fragments are then optionally modified with appropriate side chain reagents for the characterization of the folding process during biosynthesis and cotranslational modifications by the length of the biosynthesized mini intermediates by the regulating use of the required amino acids with or without isotopic labeling is achieved and the timing of formation of such mini-produced intermediates according to their length ratios for the entire amino acid sequence to be redefined.

The chosen way for modification is defined according to the invention below:
The single modification: it is defined as the selective modification of a single type of amino acid residues with a single reagent under optimized reaction conditions,
The multi modification: it is defined as selective modification of the residues in more than one kind of amino acid with a single or more than one type of reagent by changing the reaction conditions. It can also be in a single or in several batches first performed separately and then mixed together.
The internal cross-linker modification: it is defined as selective internal double modification with the bifunctional reagents at regulated reaction conditions in varying embodiments.

The known methods for the modification to be applied are diverse and, depending on the specific time scale of protein folding for the appropriate modifications are available. The choice of method decides according to the invention of the modification speed rate, which mainly depends on the reaction rate of the side chain reagents and the properties of the modified proteins. The methods can, therefore, depending on the reaction speed of modification be divided into three groups:
The modification rate with a time scale between nano-, micro- to milliseconds. This is useful for studies of protein folding with a time scale of miliseconds to seconds. These fastest phases of folding include the formation of hydrogen bonds, the formation of the secondary structure elements and the hydrophobic collapse of the polypeptide chain. These fastest modifications are possible with for example use of the specific side chain reagents or by supply of the photochemical and thermal energy for the isomerization of proline and aspartic acid and the deamidation of asparagine be achieved.
The modification rate with a time scale of microseconds to a minute. By use of microwave technology modification reaction rate can be achieved that are applicable for the study of protein folding with a time scale of milliseconds to minutes. Its about the fast folding phase and the early stage of development of different pathways associated to different intermediates.
The modification reaction rate with a timescale of milliseconds to minutes. This is useful for studies of protein folding with a time scale of seconds to hours or days, which is usually the slow phase of protein folding and the formation of pathways associated intermediates, formation of relative stable compact structures of molten globules and the further folding of intermediates to the native state with minimal energy level.

The modifications in the time scales for the fast phase of folding can be used in the respective reaction mixtures as needed according to the invention made with quenched-flow, stopped-flow and continuous-flow methods and turbulent mixing technique in connection with the microwave-mini apparatus and the spectroscopic detection. The modifications for the slow phase of the folding can be performed using conventional methods. The basic criterion for a successful modification of the refolding protein is whether all that modified and trapped intermediate products are presented by structurally relatively stable intermediates which can be separated with individual characteristics.

According to the invention reagents selected for modification include following examples:
The known and not known, but acting denaturing agents of proteins,
The known and not known, but acting agent for reduction of disulfide bonds,
The known and unknown but acting reoxidation reagents including their variable components and compositions specific for the modification of the disulfide-containing proteins,
The known and unknown, but functionally identical side chain specific reagents, with and without isotopic labeling,
The known and unknown but functionally identical reagents with the fluorescence label, spin-labeled reporter groups preferably of small molecular reagents with no effect of charge change on the protein, in particular the known reagents for DIGE (Difference gel electrophoresis),
the known and not known, but functionally identical zero-, homo- and hetero-bifunctional cross-linker reagents for internal labeling, including the reagents for photolabeling,
The known and not known but the functionally identical reagents for biotinylation.

According to the invention needed and selected auxiliary substances for stabilization, improvement and increase the native folding efficiency during modification and/or oxidation, especially large proteins include the following:
The known and not known, but the functionally identical reagents as foldases and chaperones named folding factors
The known and not known, but equally acting auxiliary substances for the optimal renaturing of the biotechnologically produced therapeutic proteins used for the reduction of misfolding, used for the decrease of aggregation and/or to increase of the thermal stability of the applied biological and chemical substances. They find appropriate applications in all embodiments of the invention for protein modification.

For performing the needed modifications in different procedures and embodiments the means according to the invention are used in the form of series assay kits, special laboratory equipment and, where appropriate, specific software. To promote the separation of modified proteins of modification reaction solution, the proteins can be according to need first chemically immobilized on a substrate, then after modification by rinsing of other components of the reaction separated, followed by chemical or photochemical cleavage freed from the substrate and for the further separation of the intermediates are provided. This applies to all proteins to be modified.

4. Separation and Quantification of the Intermediates and Two-Dimensional Representation of their Hydrodynamic Sizes and Amount of Material as a Function of their Time of Formation.

All these above-described modifications of the intermediates can be separated according to the invention in two ways according to their reached hydrodynamic sizes and quantities separated manually or according to the invention automatically quantified and classified by their hydrodynamic size or, where appropriate thermodynamic parameters and quantities as a function of their time of formation and the 4 phase multi-folding pathways model are presented according to two dimensions. A fingerprint profile of a protein folding relating to different folding phases of different pathways can be generated thereby.

The first implementation is based on the electrophoretic methods. This form of implementation refers to the preferred capillary electrophoresis and refers to the special native polyacrylamide gel electrophoresis modified by the inventor, which is preferably aimed for the global and hydrophilic proteins and the type, size and quantity of the treated proteins in the form is made of variable designs. The hydrodynamic sizes of the intermediates and their quantity are here for example, after the two-dimensional electrophoresis directly on the gel in the form of bands with different staining intensity against their time of formation submitted and prepared for the scanning and digitization for further investigations. The same gel bands, arising at different times and having the same hydrodynamic size or same gel band, represent here the same intermediates.

In the case that more than one intermediate is present in a single band, the intermediate from the bonds of the gel are electroeluted and then with the constructed by the inventor microcolumn chromatographically separated according to their differences in the distribution of charges, hydrophobicity and hydrophilicity on the molecular surface and then spectrometry with DLS (dynamic light scattering), SLS (static light scattering), CD, fluorescence is applied for differentiation.

This inventively modified native polyacrylamide gel electrophoresis is aimed to increase the separation ability between the individual intermediates to achieve the maximum separation efficiency and shorten the duration of electrophoresis, in contrast to conventional protein separation of the subtle differences of the hydrodynamic size of the of individual proteins derived intermediates to differentiate. It comprises at least five technically related improvements.
First, the connection of a delivery system of the buffer solution for appropriate regulation of the buffer concentration, composition and the pH value during electrophoresis either by embedding a permeable container which is filled with the desired buffer solution and is located next to the buffer reservoir to the cathode or by a buffer distribution device, which is located in the buffer reservoir to the cathode and connected by a thin tube with the outer vessel. This enables the desired buffer solution according to the desired speed to be fed continuously or discontinuously and dosing.
Secondly, the dynamic control and optimization of the separation resolution by the regulating during electrophoresis supplied buffer solution. The settings made in the buffer concentration, composition and the pH value leads to an increase of the difference of the charge and polarity between the intermediates of a protein and the ion current. The improved resolving power here is not only based on charges and forms of a protein alone, but also on the newly introduced differentiator factors, effectively determine the dynamically changing charge and polarity distribution of the intermediates and their diverse interactions with the additional ion current.
Thirdly, the application of pulsed electrophoresis by brief increase in voltage, short changing the polarity of the buffer components and/or their concentration and short polarity reversal combined with the highly hydrophobic counterions for focusing the single band and enlargement of the removal of the Intermediate bands.
Fourth, the use of variant gel compositions and shapes, such as by the porosities gradient gel for separation of intermediates of proteins in particular large size
Fifthly, for suppressing the thermal effects caused by diffusion of the intermediates between the bands the temperature of performed gel electrophoresis should not exceed 10° C., wherein directly the electrophoresis equipment is either in a sufficiently effective cooling thermostat connected or according to the invention is assembled into ice containing cooling reservoir from the Peltier cooling plates and a circulating liquid cooling existing apparatus for simultaneous heat removal from the gel and heat dissipation can.

The second form of implementation is designed by the inventor of the mono and multi column liquid chromatography including miniaturized field flow fractionation (FFF) and the necessary micro column electrophoresis coupled with the spectrometric differentiation of hydrodynamic size, preferably with DLS and SLS. They are suitable for the separation and differentiation according to the intermediate sizes of all proteins, but is mainly addressed to the non-electrophoresis method suitable proteins, for the highly acidic, strongly basic, hydrophobic and membranes proteins, including the very large proteins, their intermediates with gel electrophoresis can not be separated effectively. By spectrometric differentiation according to the hydrodynamic size or possibly thermodynamic parameters of each individual micro-vessels separated intermediates are assigned as function of their time of formation for the further steps in two dimensions usually arranged in a microtiter plate and kept in the database. Under mono column liquid chromatography is meant the preferred use of each as desired or required, filled with different separation media or the individual microgel filtration column or micro field flow fraction canals in connection with spectrometric examination for direct determination of the order of the hydrodynamic size of it separated in each of the individual eluates intermediates.

The multi column liquid chromatography is defined as micro-column combinations as desired or required, in series and/or in parallel from gel filtration, hydroxyapatite, hydrophobic, ion exchange, Reverse phase and affinity chromatography including the micro field flow fraction channels for the specific isolation of the intermediates, with mainly very similar hydrodynamic sizes, either belong to the same or different folding routes.

The hydrodynamic variables of isolated Intermediate are usually differentiated with multi column chromatography with micro vessels in serial or in microtiter plates spectrometrically first, then placed in the order of their size and kept in the database and then classified in two dimensions as a function of their time of formation.

The coupled spectrometric differentiation of hydrodynamic sizes of the separated intermediates in different individual micro-vessels and their quantification is preferably carried out with DLS (dynamic light scattering) and SLS (static light scattering) and this will continue with fluorescence, UV (ultraviolet radiation)/VIS (visible spectrum), CD (circular dichroism), NMR (nuclear magnetic resonance-resonance), Fourier Transform Infrarot and ESR (electron spin resonance), etc., which are offered in various forms in commercial supplements.

The specific differentiation of intermediates in single sample portion, which are distributed either in different hydrodynamic sizes or very similar to the hydrodynamic size but may belong to different folding pathways can be made with the inventively modified DLS and SLS devices, their differentiation capacity by at least one of the following additional functions can be improved:
Increase the differentiation resolution by extended measurement time,
Tm (melting point) differentiation by the program controlled by the gradual increase in temperature,
Change in concentration of the sample into individual micro-vessels or in microtiter plates by dilution or by the built-in vacuum evaporation or concentration aeration fulfilled,
Change in the pH profile and buffer system by Microautotitration or manual pipetting the desired buffer composition,
Determination and evaluation of intrinsic viscosity and the zeta potential of the samples for further differentiation of the continuous structural intermediates,
Change in biorheologic properties of the samples by supplying energy or—removal in the form of irradiation, heating and cooling, ultrasonic, microwave, electric field and magnetic field,
Assessment and arrangement of the intermediate thus differentiated by specially developed software.

With improved functions of the DLS for the distribution representation of the hydrodynamic sizes of the intermediates, a fingerprint profile of the folding of a protein which presents the operation of the construction of the folding channels in accordance with the invention defined phase of folding, with in coupling of an instrument of quench-flow or stopped-flow tandem Mixer determined under defined conditions and using the graphics software can be visualized. Here, the optimal completely unfolded protein is induced by the first mixer to refold, then after various time intervals by means of the second mixer dynamic modification starts and during which the DLS measurements to the at these time intervals resulting distributions of the hydrodynamic sizes of the intermediates to subjected to determine.

This liquid chromatography coupled with spectrometric differentiation of hydrodynamic sizes, depending on the physicochemical properties of the protein and the objectives of each investigation conducted by the inventor as defined in the micro, analytic and semi-preparative scale.

The implementation in micro-scale needs very little protein and the rapid treatment of less than 100 μg protein serves containing micro approach of the protein modification and is carried out in parallel with the micro gel filtration column constructed by the inventor in the individual, or in serial or parallel ports.

The eluates are collected mostly in the microtiter plates and provided by the spectrometric confirmation of their hydrodynamic size and concentration differences for the proteolytic cleavage. The implementation of the analytical scale needs up to 1 mg of protein and is specifically aimed for the separation of intermediates, which because of similar hydrodynamic size of the micro-scale implementation cannot be completely removed or their folding pathways identity are to be differentiated further. The separation of the intermediates in this scale is done with the multi liquid column chromatography and provides adequate protein material for the repeating DLS provisions spectrometrically structural analyzes the subsequent proteolytic fragmentation and other necessary investigations.

The performance in the semi-preparative scale requires more than 1 mg of protein and is based on the same conception of the separation which has previously been successfully demonstrated in the micro- or analytical-scale, and is used primarily for preparing samples for NMR or crystal structure determinations of separate intermediates, in order to differentiate the folding pathways of the intermediate by differentiating the individual characteristics of structural change in more detail. This implementation can be done with the help of commercial products that are state of the knowledge of chromatography, and can be prepared accordingly.

The two modes of application can according to the invention be standardized by selective compilation of the apparatuses which are state of knowledge, such as electrophoresis, liquid chromatography, electrochromatography, field flow fractionation and spectrometry preferably with DLS and SLS standarized, partially or completely automated, miniaturized and with other steps of the invention process online or offline be connected.

5. Fragmentation of Intermediates by in-Gel Digestion or in-Solution Digestion

Fragmentation is the first step for the further differentiation of the exact identity of the folding pathways of the intermediates. The fragmentation of electrophoretically and chromatographically separated intermediates, whose hydrodynamic size and time of formation have been set, preferably takes place by enzymatic digestion with trypsin. Besides trypsin other endoproteases like Lys-C, Glu-C and Asp-N can be used, which are usually used in the case of possible protease resistance against trypsin caused by modification and for preservation of favored and additional cleavage sites. The exoprotease and chemical cleavage as an alternative to MALDI-TOF-MS/MS and MALDI-TOF-PSD (post source decay) serve the hydrolysis of the amino acids from the N- and/or C-terminus of the enzymatically cleaved fragments in order to differentiate fragments with the same molecular weight. All proteolytic fragmentations can be performed manually or with a commercial digest robot, or by the inventor specifically designed microwave digestion apparatus with an average throughput of samples. The fragmented samples which are in microtiter plates can be brought to the online or offline coupling with the mass spectrometer.

The electrophoretically separated intermediates which are separated in gel bands after various time intervals are stained by the Coomassie-brilliant blue or silver staining and are displayed on the two-dimensional gel, photographed and scanned to be digitized. The quantitative evaluation of staining intensities and quantification of intermediates made with the densitometer. The kinetic relationships between the intermediates can this be interpreted qualitatively with the appropriate software. The individual or with different fluorescent dyes labeled and separated by gel electrophoresis intermediates are detected with a fluorescence scanner and digitized. If a band contains more than one intermediate, the intermediate mixture is first chromatographically separated with the microcolumn and then it will be fragmented separately. All intermediates in the bands are separated according to the invention either manually with the specific tool or with a gel bands picker automatically and they are cleaved with the standard method or with the novel digestive apparatus in multifunctional microplate for cleaved in-gel digested enzymatically into fragments. The additional exoproteolytic or chemical cleavages occur in the other solutions from in-gel digestion according to the first mass spectrometric studies.

The fragmentation of the chromatographically separated intermediates can be done either with the standard method for in-solution digestion in microtiter plates, or by analog to the last step of the handling of the gel electrophoresic separated intermediates by the inventor specifically for in-gel digestion constructed digestive apparatus.

### 6. Mass Spectrometric Detection of Fragmented Intermediates

Mass spectrometric detection is the second step for a more precise differentiation of the folding pathway identity of each intermediate. The selected methods include ESI-TOF-MS (electrospray ionization-time of flight mass spectrometry), MALD-TOF-MS (matrix-assisted Laser-desorptions/-Ionisations-Flugzeit-Massenspektrometrie) MALDI-TOF-MS/MS (tandem mass spectrometry) and MALDI-TOF-MS-PSD (post source decay-mass spectrometry), etc.

Mass spectrometric detection of the intermediates is fragmented by the measurements of the molecular weights of all the individual fragments and bound by disulfide bridges and/or crosslinker connected large fragments. All collected data are stored in a database.

This database contains a theoretical analysis of fragments, of all possible intermediates and all resulting fragments and including large fragments that presents fragments that are interconnected, according to the invention with commercial or specially developed software designed and stored.

### 7. Determination of Folding Pathway Identity of Intermediates

The crucial step for determining the folding pathway identity of intermediates is the comparison of mass spectrometric information collected on the number and mass of a few small fragments and those which are bound by disulfide bridges and/or cross-linker with the data stored in the theoretical fragment mass detection pattern. Here, the above explained criteria based on the theory of evolution of protein and protein folding kinetics, are needed to complete the definition of the folding pathways identity of the intermediates. The characteristics of each fragmented intermediate based on modification are detected by the comparison and marked with a specific title. The separated intermediates, which each come from different times are also compared and selected depending on the individual characteristics of the names. With that table is automatically created using special software. In this table in the first column the hydrodynamic size decreases from top to bottom, and specified time of formation in other columns is increasing from left to right with time intervals.

All intermediates are defined in table according to their hydrodynamic size, time of formation and, if necessary amounts. In the other columns of the table the different markings or labels are specified for all intermediates. The same labeled intermediates can be classified into groups with the natural numbers. Assigned by a number of group memberships of all intermediates are listed in the one before the last column. If one intermediate belongs to an intermediate group with such a natural number, its membership folding pathways is determined by this number and entered in the last column.

The separated intermediates whose folding pathway identity until then is not clearly defined can be further analyzed through the use of liquid chromatography in analytics and semi-preparative scale described in the fourth step and coupled with improved DLS and SLS devices that are if necessary with structural and thermodynamic studies of CD, UV, NMR and fluorescence studies etc., can be differentiated with respect to their folding pathways identity.

This will show that the folding pathway identity of an intermediate cannot be defined by itself but by group membership of a particular folding pathway.

### 8. Identification and Classification of the Intermediate

The identification of an intermediate is carried out by determining of its previously established hydrodynamic size, time of formation, its amount quantified and identified folding pathway identity

All intermediates can be identified according to their individual name of their group membership of a particular folding pathway. The group associated intermediates should have certain common characteristics. The features may, depending on the diversity of groups in various shapes such as in the following descriptions become apparent.

They should have a similar modification pattern. Their hydrodynamic size should decrease with the time intervals in the direction of the native structure in stages. They can have at least one significant intermediate, which has a relatively stable compact structure which may be referred to as molten globule. They can also include intermediate, whose hydrodynamic sizes are very similar and between which the intramolecular rearrangements take place. Furthermore, they can also contain the intermediates, which belong more than one group, because the road-junction, extension,—junction—and crossing must be inserted through these intermediates.

They often have the same quantified amount, which decides on the competence of the kinetic folding pathway. Intermediates which are grouped in the fast folding, often involve the native conformations, and the grouping in the slow folding intermediates include the other hand, most non-native conformations. The fast folding group usually has the fewest intermediates with little intramolecular rearrangements. The slow folding group usually has several intermediates and is almost always accompanied here by the intramolecular rearrangements of the structure, which lead to change in the microenvironment and thus also lead to changes in the modification characteristics between the intermediates. The intramolecular rearrangements often take place at the molten globule state. The intermediates subjected to intramolecular rearrangements have similar hydrodynamic sizes and are swayed by the difference attributable to the modification of the structural characteristics differentiate.

All intermediates assigned to different groups can further be classified according to their own characteristics of the groups into different folding pathways. Thus, the routes for all intermediates is differentiated and identified. The hydrodynamic size, the time of formation, the quantified amount, the folding pathways identity and where appropriate, the NMR structures of all intermediates are defined here in tables and provided for the subsequent characterization of the folding process.

### 9. Characterization of the Folding Process

The characterization of the folding process takes place according to the invention by the parallel representation of all is the folding pathway grouped intermediates in a two-dimensional coordinate system for the simplest folding process, their hydrodynamic variables entered in each case against the time of formation and a systematic evaluation of these series. If folding process contains more than one way, the characterization of the folding process can be shown on the same principle in a multidimensional coordinate system including the additionally inserted coordinates as a folding channels or folding pathways.

In the presentation it is shown that the process of protein refolding pass through four phases, namely the super fast folding to the formation of seed structures of the folding pathways, the formation of the folding pathways or channels, the subsequent passage through the folding along the constructed pathways or channels and the more extensive restructuring by intramolecular rearrangements to complete the native structure of the protein. The characterization of the folding process of a protein mainly through the entire characteristics of all intermediates occurring in the first two phases of folding and the formation of folding pathways or canals during these 2 phases can be named as a fingerprint of the protein folding process. The different folding pathways as parallel events at different speeds, including the way-junction, extension, intersection, crossing, and the traverse, conjunction can be seen in the clearly. By this it is possible for all intermediates to identify folding pathway identity, their kinetic process, their percentage contribution to the folding, the process of misfolding, the origin and the course of the intramolecular rearrangement, the native and normative disulfide bonds, the sequence of the native disulfide bonds and it is possible to identify folding rate determining intermediates. This allows the characterization of complete folding process of a protein according to the invention.

By this determined data on the change in the amount of the intermediates related to the time of formation presents the kinetics of the protein folding process and can used to provide additional dimensions for the refined characterization and visualization.

The characterization of the folding process can also be done on the three-dimensional structural level of all intermediates. Here are all the intermediates of a protein whose folding process has been characterized according to the invention, after the same principle in each case the quantity required for NMR or/and crystal structure analysis of modified absorbed, separated by liquid chromatography and the structure determinations are carried out. This semi-preparative production of intermediates can be done with the specially developed by the inventor of the process in semi-praparative scale, which is specifically geared to the efficient separation of the trapped intermediates with large concentration differences.

The characterization of the folding process with the inventive method can be extended at the functional level of a protein in different embodiments for various applications, wherein the folding process of this protein are first characterized according to the invention and then further under the influence of its own structural changes or the modified biological and chemical environments for investigating modified functionality and activity of this protein is characterized. This extension includes, for example, the characterization of the process to simulate dynamic in-vitro post and co-translational modifications, the procedure of dynamic modifications in simulated in vitro protein folding and modeled the process of in vitro biosynthesis of the protein.

### 10. Graphical and Visual Representation of Protein Folding Process

If a folding process of a protein is characterized according to the invention, all involved intermediates, each with its 4 individual characteristics, namely the hydrodynamic size, the time of formation, folding pathway identity and the percentage contribution to refold can be presented in a multi-coordinate system, 3- or 4-dimensionally digitized. For example, the energy states of the intermediates according to their hydrodynamic size and the gel bands is ploted as the y-ordinate, the time of formation as the x-ordinate, the folding pathways identity or channels defined according to their folding pathways identity as the z-ordinate and, where appropriate, the percentage contribution to the refolding as an integrated 4th dimension. This makes it possible to perform graphic visualization and animation in diverse ways with appropriate computer graphic software in order to present the folding process.

All folding events, which are characterized by the use of the inventive method in various embodiments, the same principle can be applied to visualize multidimensional and/or animate.

From the transformability of all of the four characteristics defined ordinates and from new combinations of these ordinates it is shown that the there are a many possibilities how to present the folding process in diverse multi dimensional coordinate systems, and that all the above-mentioned characterized embodiments and processes of protein folding can be presented in many different ways. Under the transformability of the four ordinates is meant for example the substitution of hydrodynamic size or gel band with the energy state of the folding, the time of formation with the configuration decrease in the folding, the folding pathways identity to the directional folding direction, the amount of the intermediate with the percentage contribution of the folding. Under the combinability is meant a hybridization of the ordinates for example, time of the folding and the amount of the Intermediates lead to the kinetics of folding. The energy state and the amount of the intermediate result in the contribution to the folding, and the pathway direction and the amount of the intermediate result in the competence of the folding, etc.

The resulting relationship between global structure and energy states of the intermediates of a refolding protein may be quantitatively presented in the variety of its forms in a multidimensional energy landscape model. The folding pathways of folding process can be presented in an octant of the coordinate system, for example, as represented by ski trails leading from top towards valley or in 2 or 4 octants defined depending on the contribution to the folding, as the traces of a high level to the lowest point of the valley and the popular funnel model (Schultz, 2000).

It will be appreciated that the inventive method is extended by determination of the 3-dimensional structures by NMR or x-ray analysis of all intermediates or only the significant intermediates with an additional 5th characteristic for the identification of the intermediates and that the folding process of the proteins characterized on the basis of the 5 characteristics of the intermediates, is characterized namely the hydrodynamic size, the time of formation, folding pathway identity, the percentage contribution to the refolding and the three dimensional structure and represented in a multidimensional coordinate system with at least 5 ordinates and visualized in all diversity.

### Advantages of the Inventive Method

The invention is capable of repealing the problems of the prior art according to the features of patent claims of the present invention. The advantages of the inventive method compared to the prior art are summarized in the following sections.

The inventive method is based on at least 4 individual and digitizable characteristics of the intermediates, namely, the hydrodynamic size, Time of formation, Folding pathway identity and amount. Therefore, numerous methods of bioanalysis can be used optimally and efficiently through flexible combination of technology. This enables, that the characterization of the folding process requires only a little amount of protein material, is easy to use, is time saving, can be standardized and routinely performed.

This implementation can be extensively automated and miniaturized. The inventive method is based on the diversity of modifications for the determination of the intermediates affiliation to a particular folding pathway. The variety of possibilities for modifying the refolding protein ensures that each resulting intermediate is modified accordingly and thereby individually marked and therefore distinguishable from others. This includes all proteins.

Thus, for example, the small and big proteins, the proteins with and without disulfide linkage, the strong acidic and strong basic, the hydrophilic and hydrophobic, the membrane- and multidomain proteins are covered. The separation of the intermediates and the determination of their hydrodynamic size, the time of formation and the amount can be fulfilled effectively, both directly through the inventive gel electrophoresis for the hydrophilic and globular proteins, and by liquid chromatography and subsequent spectrometric investigations as preferably by DLS (dynamic light scattering). In so doing all proteins, as listed above, can be involved in examination.

The structured proteolytic system of trypsin, besides Lys-C, Glu-C, Asp-N and exoprotease as well as chemical cleavage coupled with MALDI-TOF-MS/MS (tandem mass spectrometry) and MALDI-TOF-MS-PSD (post source decay mass spectrometry) can ensure that after their proteolytic treatments all intermediates exist in appropriate fragments with suitable modifications for the identification of fragment patterns can be detected spectrometrically and for further work appropriate digitalized.

The hereby characterized process of protein folding can be represented in a variety of multi-dimensional depictions and a quantitative visualization of the relation between spatial structure and energy landscape of protein folding intermediates, corresponding to the funnel concept (Schultz, 2000), will be allowed.

The advantages of the inventive methods are also in its various applications. In a first preferred application of the invention, the method is provided for the characterization of the folding of all types and sizes of proteins, the elucidation of the folding mechanism including the folding pathways, the process of misfolding and the process of intramolecular rearrangements and also for the detection of the causes of certain proteopathies.

In a second application of the invention, the method is used for investigating and evaluating the changes in activity and function of a refolding protein due to its substrate in order to improve or optimize its biotechnological production. For this purpose the folding process of the proteins to be examined is characterized with and without substrate at varying molecular environments according to the invention, and the results are compared and evaluated.

In a third application of the invention the method is used for protein engineering, whereat for example, the modifications based on the findings of the characterized folding processes, the redesign and the fusion of proteins and the subsequent changes in functionality and activity of a protein due to the redesign are analyzed examined and evaluated according to the invention.

In a fourth application of the invention, the method is used for the dynamic characterization and quantification of the processes of in vitro-simulated biosynthesis and possibly occurring co- and posttranslational modifications such as an optimization of the conditions of biotechnological production of in vitro post-translationally modified protein therapeutics and the scanning of the chemical and biological auxiliary compounds or inhibitors of the co- or post-translational modifications. Thereby the characterization of the dynamic processes during the in vitro simulation of the co- and posttranslational modifications is performed according to the invention and the results are subjected to the corresponding comparison and evaluation studies.

In a fifth application of the invention, the method is used for the dynamic characterization of the refolding process of the proteins during their in vitro simulated, known in vivo post-translational modifications for process development of its biotechnological production. The characterization of these processes follow the steps defined according to the invention. It is possible to immobilize the proteins to be investigated in combination of the technology of protein immobilization and protein chip preparation on the support, to separate them after simulated post-translational in vivo modifications from the chemical, enzymatic or the cell extract containing reaction solution, then cleave the proteins from the support by thermal, photochemical, chemical or enzymatic cleavage and use them in further steps of the inventive process.

In a sixth application of the invention, the method is used for the search for biological or chemical agents in the protein folding, wherein the selected biological or/and chemical inhibitors and/or auxiliary proteins are incorporated in the inventive characterization of the refolding of the desired protein.

In a seventh application of the invention, the method is used for investigation of the effect of foldases or chaperones on protein folding, wherein the impact is defined by comparison of the inventively characterized folding processes with and without the foldases or chaperones to be examined.

In an eighth application of the invention, the method is used for searching for novel pharmaceutical agents, that are belonging to the biological and chemical inhibitors of foldases/chaperones including proteins that influence protein degradation, wherein the inventive characterization of the folding process of the protein to be examined is achieved in parallel experiments containing chaperones or foldases with and without the inhibitor candidates of the chaperones or foldases. Through the subsequent comparison of the folding processes, the effects of candidate compounds on the inhibition of foldases/chaperones and protein degradation are confirmed.

In the ninth application of the invention, the method is used for the investigation of controlled self-assembly and polymerization of the polypeptides or proteins during their refolding process on behalf of the development and production of nano-protein materials, wherein the initial process of self-assembly and polymerization of a protein to be examined is characterized according to the invention in the presence of biological and/or chemical factors.

In a tenth application of the invention, the method is used for searching for pharmacological chaperones against proteopathy, whereby the effect of certain biological and/or chemical substances which specifically bind to unfolded proteins, which improve the folding process and stabilize the protein structure, or mask the hydrophobic domains of misfolded proteins and increase the solubility and therefore prevent aggregation of unfolded proteins, on the refolding mechanism of proteins causing proteopathy is determined by the inventive characterization.

In an eleventh application of the invention, the method is used at one hand for characterization of the process of refolding of PrP^{c} (Prion Protein cellular) to PrP^{Sc} (Scrapie prion protein; pathogenic form of prion protein) including the following aggregation and at the other hand the reversal of the PrP^{Sc} zu PrP^{c} including the degradation of aggregations to clarify the pathogenesis and enables the search for treatment options and possibilities for prevention.

The folding processes of a prion protein are employed in parallel experiments with biological and/or chemical substances influencing the folding process under the regulation of destabilizing conditions such as temperature, pH and ionic strength according to the invention and are compared with its previously characterized folding process without the active substances.

In a twelfth application of the invention, the method is used for the dynamic characterization of the folding process of a targeted protein for the study of the protein aging due to isomerization, deamidation and racemization and for the study of protein degradation caused by radical action, oxidative stress and environmental. Hereby the specific protein is subjected during its refolding process either to a catalytically accelerated isomerization, deamidation and racemization by the supply of photochemical and thermal energy or to a radical-initiated action, oxidative stress and modification by environmental influences. The hereby altered folding process is then characterized in further steps of the inventive method and is compared with the folding process without this characteristic influence factors.

In a thirteenth application of the invention, the method is used for the classification (taxonomy) of the proteins and for the study of protein evolution to a new level of dynamic protein folding, where the inventive characterized process of folding as an individual fingerprint of each protein provides the functional and evolutionary relationships of proteins and can be integrated as an additional criterion in the previous classification, based on the structure, topology, homology and evolutionary relationship.

In a fourteenth application of the invention, the method is used for dynamic characterization of the folding process of proteins during complex formation with nucleotides, glycosids and lipids to study their formation process and the accompanied changes in activity and functionality, and to find chemical and biological substances acting on the complex formation. Hereby the folding process for example of a protein and its protein-nucleic acid complex are characterized initially each isolated according to the invention and this protein and the corresponding nucleic acid are then brought together during their refolding at time intervals in successive experiments for complex formation and are subjected repeatedly to the novel characterizations, comparisons and evaluations with and without supply of the substrate of the protein-nucleic acid complex under varying chemical and biological factors. In doing so the additional masses of the nucleic acid-, lipid- and carbohydrate fractions during creation of the fragment mass pattern will be counted accordingly. For the qualitative and quantitative determination of the non-covalent fraction of these proteins the batches, after the proteolytic cleavage and before mass spectrometric measurements, are subjected at first to a chromatographic separation and then to a spectrometric determination of the hydrodynamic sizes and masses of the fragments.

In the fifteenth application of the invention, the method is used for the diagnosis and prognosis of diseases caused by protein misfolding, whereby the changes in the folding process of the disease-related proteins are characterized, presented in the form of a fingerprint profile of the folding process and are defined as criteria of diagnosis and prognosis of certain diseases.

In a sixteenth application of the invention, the method is used for the dynamic characterization of the initial process of the aggregation of the same proteins or different proteins during their refolding between the folding proteins or between the folding and native proteins to elucidate the mechanism of aggregation and search for chemical and biological inhibitors of protein aggregation, wherein the folding process of the protein to be examined is characterized, compared and evaluated according to the invention first without and then under the influence of other proteins and/or chemical and biological inhibitors in different molecular environments respectively.

In a seventeenth application of the invention, the method is used for the dynamic characterization of the folding process of the interactions between the refolding proteins and/or between the refolding and native proteins, for the analysis of the conformational behavior and the catalytic properties of specific proteins at the level of its refolding, for the search for therapeutic target proteins enabling a rational design of drugs and for the optimization of biotechnological processes, wherein the designed folding operations of the protein to be examined are characterized, compared and evaluated according to the invention, first without and then under the influence of other proteins in optimized molecular environments respectively.

In the eighteenth application of the invention, the method is used for the dynamic characterization of the process of antigen-antibody reaction and the degradation process of the antigen-antibody complex for optimizing and rationalizing the antibody engineering. Here, the newly designed antibodies and antigens are subjected to the inventive characterizations, the comparison and analysis first singly, then together in an optimized physiological and biochemical molecular environment without and/or with the chemical and biological factors to investigate the selectivity, specificity, affinity, folding efficiency, thermodynamic stability, pharmacological kinetics and biotechnological productivity of the redesigned antibody and antigen.

In the nineteenth application of the invention, the method is used for the characterization of the folding process of in vitro biosynthetic produced and perhaps co-translationally modified proteins to investigate folding of the protein initiated during its nascent biosynthesis and to search for the chemical and/or biological substances that influence the initiated folding of the protein, wherein the cell-free and in the batches synthesized protein fragments with different lengths obtained under optimized biological and physicochemical factors by regulating supply of the necessary amino acids with or without isotopic labeling and referred to as mini-intermediate, first are collected together and then are specifically separated chromatographically followed by the characterization and comparison of their folding processes according to the invention. Here, the formation times of the mini-intermediates are redefined according to their length ratios in comparison to the whole amino acid sequence.

In another application of the invention, the method is used for the construction of databases based on the characterized folding processes of proteins and thus introduced applications, that are available as service centers for the further development of new applications.

These uses include, for example, new modifications, redesign, and fusion of therapeutic proteins based on the results of the characterized protein folding and the prediction of the fingerprint profile of the folding process of newly constructed proteins.

A particular advantage of the invention is that the embodiments of the method can be extended depending on the objective by individual combination and addition of methods and techniques.

The products of the process, characterized by the diversity of modified intermediates of the protein and the thereby in different applications in different molecular environments under different physicochemical and biochemical factors determined and in a database systematically collected findings of the folding processes of all characterized proteins, can be designed and used for the screening of chemical and biochemical agents on protein folding, for the improvement and optimization of biotechnological production, for the optimal restoration and preservation of the target proteins, for the efficient modification, for the re-design, for the rational fusion proteins and to improve their structural function, activity and pharmacokinetic and pharmacological properties.

The inventive method involves the use of the new materials, namely proteins after optimal and complete refolding and in time intervals accomplished varied modification, in the form of intermediates, each with at least 4 independent characteristics. This according to the invention in various embodiments recovered protein materials are used for the characterization and multi-dimensional representations of their folding processes carried out in varied environments under different physico-chemical and biochemical conditions to elucidate the mechanism of folding, misfolding, aggregation, interaction, self-assembly, polymerization, aging, wear and the biosynthesis of proteins, improvement of protein activity and -functionality, optimization of the biotechnological production, development of nano-protein materials, enrichment of the protein taxonomy and to enable the search for novel biological and chemical agents and protein therapeutics with their activity based on their influence on protein folding.

### Means for Performing the Method

To carry out the inventive process the means in the form of assay kits, special laboratory equipment and specific software are used. The inventive process can be accomplished either manually step by step, or by means of partial and/or fully automated and miniaturized devices, which are specifically developed and manufactured according to the inventors conception and design.

### Assay Kits

The novel series of assay kits mainly consist of conventional chemicals, materials, components and instructions for use according to the invention. These products result from the development, optimization and standardization of experimental conditions and handling of each experimental step until means of systematic characterization of the refolding of all kinds and types of proteins were developed. They are used depending on the implementing steps, methods, embodiments and applications of the inventive method. The different types of Assay-Kits for different applications are described and classified in the following:
for the production of optimal unfolded proteins with maximal hydrodynamic size,
for the dynamic modification of the immobilized proteins during their refolding process each with varied side chain reagents,
for the dynamic modification of the refolding protein with one or more fluorescent dyes,
for the dynamic modification of the refolding proteins with isotopic or spin labeled side chain reagents,
for the dynamic modification with biotin tags during the refolding of a protein,
for the dynamic modification of the disulfide containing proteins, each with varied side chain reagents and for the preparation of their refolding fingerprint profiles,
for the dynamic single, multi and/or internal crosslinking modifications of the refolding proteins without disulfide linkages, each with varied side chain reagents, including the zero-length-, homo- and hetero-bifunctional reagents and the reagents for photo affinity labeling and the creation of their refolding fingerprint profiles,
for the dynamic modification of the refolding globular and hydrophilic proteins, each with varied side chain reagents and the creation of their refolding fingerprint profiles,
for the dynamic modification of the strongly hydrophobic proteins and membrane proteins during their refolding process, with varied side chain reagents and the preparation of their refolding fingerprint profiles,
for the dynamic modification of the strongly acidic or strongly basic proteins during their refolding process with varied side chain reagents and the preparation of their refolding fingerprint profiles,
for the dynamic modification of isolated protein domains of the mutually independent multi-domain proteins, during its refolding process, with varied side chain reagents and the preparation of their refolding fingerprint profiles,
for the dynamic modification of interdependent and compound multidomain proteins during their refolding process and for the preparation of their refolding fingerprint profiles,
for the dynamic characterization of a refolding protein with its substrate or its potential substrate in the batches under different selected physicochemical and biochemical conditions for the investigation and verifying of changes in activity and functionality during its refolding process,
for the dynamic characterization of the refolding process of redesigned or biotechnologically modified proteins or fusion proteins and the analysis of the changes in function and activity caused by this protein modification, based on the comparison with the data from the characterization of the refolding process of the mentioned protein,
for the dynamic characterization of the mechanism of the in vitro simulated biosynthesis and possibly occurring co- or post-translational modifications of a protein in cell-free approaches under selected physicochemical and biochemical molecular environments for optimization of the conditions for biotechnological production of the in vitro post-translationally modified protein therapeutics,
for the dynamic characterization of the refolding process of proteins during their selected, in vitro simulated, post-translational in vivo modifications in biological or cell-free approaches under selected physicochemical and biochemical conditions for process development of its biotechnological production,
for the dynamic characterization of the refolding process of proteins under the influence of selected biological and/or chemical substances for the investigation and verification of potential drugs on protein folding,
for the dynamic characterization of the refolding process of proteins in experiments with selected foldases or chaperones for investigation and verification of its auxiliary effect on protein folding,
for the dynamic characterization of the refolding process of proteins in experiments with selected foldases or chaperones and their potential biological and chemical inhibitors for investigation and examination of their inhibitory effect on protein folding,
for the dynamic characterization of the refolding process of polypeptides or small proteins in experiments with additional biological and/or chemical substances for controlled investigation of self-assembly and polymerization,
for the dynamic characterization of the refolding process of a proteopathic protein in experiments with potential biological and/or chemical substances possibly stabilizing the refolding process for testing and verifying its pharmacological effects as chaperones against proteopathy,
for the dynamic characterization of the refolding process of a prion protein in parallel assays with biological and/or chemical substances with influence on the folding process under regulation of the destabilizing conditions to investigate its pathogenesis and to verify the options of a possible treatment and prevention,
for the dynamic characterization of the refolding process of a targeted protein in experiments, in each case under photochemical and/or thermal energy supply, free radical exposure, oxidative stress and the influence of altered molecular environment to investigate the protein aging caused by isomerization, deamidation and racemization and for investigation of protein degradation caused by radical action, oxidative stress and environmental influences,
for the dynamic characterization of the refolding process of proteins in experiments with molecular environments and under standardized physico-chemical and biochemical conditions optimized for the types and sizes of investigated proteins, for obtaining information on protein classification (taxonomy) and protein evolution,
for the dynamic characterization of the refolding process of a protein in experiments to investigate its complex formation with nucleotides, glycosides and lipids under influence of various chemical and biological factors and regular supply of potential substrates for the investigation of the formation process and the associated changes in activity and functionality and also to investigate the pressure exerted on the complex formation reactions by the added chemical and biological substances,
for the dynamic characterization of the refolding process of a disease-related protein in experiments under optimized and standardized physico-chemical and biochemical conditions for the preparation of its refolding fingerprint profile as criterion for its diagnosis and prognosis,
for the dynamic characterization of the refolding process of a protein in experiments without and with other proteins under varying molecular environments and regular addition of chemical and biological inhibitors (including potential inhibitors) of protein aggregation for investigation and verfication of the aggregation mechanism of the same protein or different proteins during their refolding process between the folding Protein or between folding and native proteins,
for the dynamic characterization of the process of interaction formation between the refolding proteins or/and between the refolding proteins and native proteins in the batches with and without potential substrates or factors under optimized molecular environment for the investigation and verification of the conformation and catalysis characteristics and therapeutic target effects during their refolding,
for the dynamic characterization of the process of antigen-antibody reactions and degradation process of the antigen-antibody complex in batches with chemical and biological factors for the investigation and verification of the changes in selectivity, specificity, affinity, folding efficiency, thermodynamic stability, pharmacological kinetics and biotechnological productivity of the antibodies and the antigenicity of the antigen,
for the characterization of the in vitro folding process of the biosynthetic produced and possibly co-translationally modified protein in cell-free approaches in presence of optimized biological and physicochemical factors and regulated supply of necessary amino acids without or with isotopic labeling for the investigation and control of the initiated folding process of the protein during its nascent biosynthesis and thereon acting chemical and/or biological substances,
for the production of native polyacrylamide gels and the electrophoretic separation of the protein intermediates that were dynamically modified during the refolding process,
for the electrophoretic separation of the intermediates, that were dynamically modified during the refolding process of the protein, with the ready to use polyacrylamide gels,
for multidimensional liquid chromatographic separation of the intermediates, modified during refolding of the protein, with parallel and serial disposable micro-columns and/or micro-column module,
for the separation of the intermediates, modified during refolding of the protein, with individual, parallel and serial micro-column electro-chromatography,
for buffer exchange of the intermediates, modified during refolding of the protein, with disposable micro-columns and/or disposable micro-column modules,
for the concentration of the intermediates, modified and separated during refolding of the protein, with disposable micro ultrafiltration columns and/or micro ultrafiltration column module,
for the microwave-assisted in-gel digestion of the intermediates in the specially manufactured multifunctional microtiter plates with specific tools for the manual treatment of the separated gel bands,
for the multiple in-gel proteolytic digestion and additional chemical cleavage of the intermediates in the multi-functional microtiter plates,
for the microwave-assisted in-solution digestion of the intermediates in the multi-functional microtiter plates.

Each of the deployable assay kits described above contains at least one of the following chemicals, Materials or inventive components in varied forms,

the different buffer solutions, denaturing agents, reducing agents, agents for reoxidation inclusive of their variable components and compositions, side-chain-specific reagents without or with isotopic, spin- and fluorescent labeling, biotinylation reagents, reagents for internal cross-linker labeling, reagents for gel electrophoresis, factors that act on the folding process called foldases and chaperones, potential inhibitors of foldases and chaperones, biological and chemical auxiliaries and inhibitors of protein folding and protein degradation, stabilizing agents in protein folding, specific cell extraction methods for in vitro protein synthesis and posttranslational modifications, etc.,

the different bottles, test tubes, reaction vessels, multifunctional microtiter plates, the gel bands pickers, special syringes for the preparation of the gradient gels, prefabricated native gel, the disposable micro-columns and micro-column modules for liquid chromatography and electro-chromatography, the disposable micro-ultrafiltration columns and disposable micro-ultrafiltration column modules, the supports for protein immobilisation and corresponding auxiliary software etc. . . . .

The assay kits are not limited to above-noted examples, as the new assay kits can be further developed and manufactured as needed to perform certain steps of the inventive method by combining and complementing or sharing of functional components according to the existing assay kits.

### Special Laboratory Equipment

The laboratory equipment to be used for implementing the method for the characterization of protein folding according to the invention involve
the microwave assisted quenched-flow apparatus for the dynamic re-oxidation and simultaneously modification of the intermediates,
the microwave mini apparatus that can be connected with the quenched-flow probe head, for modification of the intermediates,
the inventive gel electrophoresis apparatus for the electrophoretic separation of the modified intermediates,
the inventive apparatus for electro-chromatographic separation of the modified intermediates,
the digital gel scanner for the graphic recording and quantification of the separated and stained gel bands,
the digital micro fluorescence scanner for the graphic recording and quantification of the separated and fluorescent gel bands and in particular for the digital differentiation of the different intermediates with similar hydrodynamic size,
the gel band picker for sample preparation of in-gel digestion,
the microwave digestion apparatus for in-gel and in-solution digestion,
the automated module for serial micro-filtration columns for chromatographic separation of the modified intermediates,
the according to the invention improved DLS and SLS-measuring apparatus (dynamic and static light scattering) for the systematic identification and differentiation of the hydrodynamic sizes of the intermediates in the multi-functional microtiter plates,

### Conception and Design for the Automated Execution of the Process

The execution of the method can be automated in a machine, manufactured in accordance with the concept and design of the inventor. The machine is made of 6 functional units, each denoted as part 1 to 6 as shown in FIG. 10. Their functions are explained in detail below.

The purpose of functional unit number 1 is the isolation of the optimal unfolded protein. This functions are accomplished by the pump, the valves, the chromatographic columns or the field-flow fractionation channels and the serial, refillable and/or disposable vessels for the provision of different denaturing reagents and unfolding batches. The reaction vessels can be also provided with a thermostat. The protein already adequately dissolved in denaturing agent is first fed by the pump in an access of the second valve and is then brought to a vessel, by switching over to the first valve and the hereby ensued flow of selected denaturing solution, and is further subjected to a mixing process via ultrasound or shaking. The denatured protein, denatured according to the programmed conditions, has different structure size and is brought by the pump into the chromatography column or the field-flow fractionation channel. The hydrodynamic size and the distribution of the protein group separated by hydrodynamic size are analyzed spectrometrically by DLS and the resulting data is stored in a database. This process is repeated with different denaturing solutions under pre-programmed conditions. Then, an approach with the best reaction conditions is determined to gain the optimal proportion of unfolded protein, which has a relatively stable maximum hydrodynamic size. Finally, the optimal approach detected by the computer, is performed again and the obtained eluate with maximum hydrodynamic size of the protein is detected by DLS and introduced by the pump to the functional unit number 2.

Functional unit number 2 is for the optimization and the dynamic modification of the protein. The function carriers are analogous to the devices in functional unit number 1, but suitable for smaller volumes. The small, disposable vessel-module can also be applied for the provision of various modification reagents. The eluate delivered from functional unit number 1 is distributed to the reaction vessels. After different time intervals from seconds to minutes solutions of a variety of modification reagents is added to the eluate. This is to capture the intermediates during formation of the refolding channels. The modification batches are then applied one by one to the liquid chromatographic separation of the trapped refolding-intermediates with different hydrodynamic sizes. By spectrometric analysis of the eluate, the distribution pattern of the hydrodynamic radii for every modification approach is detected. These data is used to identify the approach with optimal modifying reagent that leads to a broad distribution of relatively stable and chromatographically separable intermediates. The optimal identified conditions are used for the dynamic quench-flow modification in functional unit number 3.

The intended use of functional unit number 3 is the dynamic quench-flow modification of the protein. The function carriers include the quench-flow-, microwave- and sample-collecting modules and the valves, etc. The optimal eluate from functional unit number 1 is directly introduced into one of four syringes of the quench-flow module. The refolding buffer solution or the reoxidation solution, the optimal modification-reagent solution, which was determined by functional unit number 2 and the stabilizing solutions are each fed into another syringe. The refolding process of the protein is started in the first mixer. The dynamic modification takes place at time intervals by mixing the solution of modification reagents in the second mixer, whereby the mixer and the delay tube are treated with a special microwave unit to accelerate the reaction rate. The intermediates, that were successively modified in portions and at the same time trapped in the delay-tube, can either be conducted through the third mixer for further structural stabilization or are passed directly to the micro-vessels of the automated sample collection module and are directed into the functional unit number 4 for further separation.

Functional unit number 4 is for the liquid chromatographic separation of the dynamically modified intermediates. The functional parts are the disposable micro-column module, the disposable ultrafiltration module, the microtiter plates, the pumps and valves, etc. In the first approach the modification batches, which are stored in the microvessels of functional unit number 4, are fed into the micro-column and are subjected to liquid chromatographic separation of the intermediates. The chromatographic separation is executed consecutively for each modification batch. The eluate is concentrated in disposable ultrafiltration modules and the separated eluates of intermediates are analyzed by DLS and are subsequently delivered to functional unit number 5. In the other approach, the modification batches are first introduced consecutively into the micro-columns of the disposable module. Then, driven by the pump, the separated and concentrated eluate of intermediates is directly applied to the microtiter plates without DLS-analysis, and is available for following off-line operations. (Sinn ist da, aber nicht wörtlich übersetzt)

Functional unit number 5 is conceived for the proteolytic cleavage and mass spectrometric analysis of the fragmented intermediates. The consecutively arranged functional parts are the online and offline digestion robot with coupling to the microwave unit, the ESI-MS, MALDI-MS, the microtiter plates, connection to the DLS detector, etc. The eluates, supplied by functional unit number 4, are first delivered to the online digestion robot for proteolytic cleavage. The thus fragmented intermediates are then introduced online to the ESI-MS for data acquisition of the number and masses of fragments of the intermediates in all eluates. The separated intermediates, which were collected on microtiter plates by the second procedure of functional unit number 4, are detected, distinguished and quantified by the coupled DLS and UV measurements, and are subsequently subjected to proteolytic cleavage in the offline digestion robot. The data collection of the fragments of all intermediates is performed by offline MALDI-MS measurements of the selected eluates in the microtiter plates. The two digestion robots are to be provided each with a microwave unit.

Functional unit number 6 is to perform three tasks, namely the setup of the application programs, the process control, data analysis and graphical presentation of the results. The functional parts include the control- and connection components, computer systems and software for automating, the analysis and representation. During setup of the selected application program a process plan is created, checked, visualized as flowchart alterable during operation, and executed automatically. The course of the process plan is visualized, as well as minuted and archived during its monitoring and control. Data resulting from DLS-UV- and mass spectrometry measurements are recorded automatically and are analyzed in many ways. All intermediates are identified here by their four characteristics and are classified according to their folding pathway affiliations. Based on these facts, the characterization of the folding process of proteins and its multi-dimensional visualization is automatically done with special software.

An advantage of this design is, that functional units number 1 to 5 can be constructed each as separate machine to perform particular steps of the inventive method. Therefore every functional unit is linked to functional unit number 6, and the functional units number 1 to 5 can be connected to each other for the automation of complex operations. Based on this conception and design, continuously miniaturization of the machines is possible.

### Special Software

The specific software used for the accomplishment of the respective steps of the inventive process includes:
the program for the classification of the protein, depending on the size of the molecular weight, the type of protein, the content of disulfide bonds, the hydrophilic or hydrophobic properties, the single domain or multidomain character and the physicochemical properties of the proteins and the hereupon based decisions on the method of separation, buffer systems and the physicochemical and biochemical conditions,
the program for the selection of side chain-specific modification reagents based on the primary and three-dimensional structure of the protein,
the program for the operation of the microwave mini apparatus for rapid modification of the intermediates in coupling with the quenched-flow apparatus,
the program for the operation of the specific improved gel electrophoresis apparatus, and the coupling with the digital gel scanner, the micro-fluorescence scanner and the gel band picker,
the program for the operation of the chromatographic separation of the modified intermediates with the disposable set of modules of the serial micro-filtration column and the micro ultra filtration column,
the program for differentiation, classification, and the two-dimensional graphic representation of the hydrodynamic size and quantity as a function of the folding process of the intermediates, based on spectrometric data,
the program for the creation of the manifold graphical representations of the refolding fingerprint profile of a protein on the basis of the measurement data from the, according to the invention defined, first 2 phases or all 4 phases of its refolding,
the program for the operation of the microwave digestion apparatus for the medium sample throughput of in-gel and in-solution digestion in coupling with gel band picker and disposable module set of the serial micro-filtration column and micro ultra filtration column,
the program for the prediction of the theoretical fragment pattern based on the primary and three-dimensional structure of the protein and the proteolytic cleavage of its modified intermediates, for the hereupon based comparisons with the spectrometrically measured data and advanced evaluations,
the program for the prediction of the theoretical fragment pattern of the in vitro post-translationally modified and proteolytically cleaved intermediates, for the hereupon based comparisons with the spectrometrically measured data and further analysis,
the program for the prediction of the theoretical fragment pattern of the in vitro simulated refolding of the modified and proteolytically cleaved intermediates in presence of chemical and/or biological factors and for the hereupon based comparisons with the spectrometrically measured data and further analysis,
the program for the prediction, differentiation and evaluation of the theoretical fragment pattern of the proteolytically cleaved intermediates with respect to the single-, multi- and internal cross linker modifications in each case with all known and not known side-chain-specific reagents with the same effect, with or without isotopic, fluorescence and spin label including the zero-length, homo- and hetero-bifunctional reagents and the reagents for photo affinity labeling and biotinylation,
the program for the classification of group membership of the identified intermediates,
the program for the determination of the folding pathway identities of the intermediates classified into groups,
the program for the graphical representation in the multi-dimensional coordinate systems of the characterized folding process of proteins,
The program for the multi-dimensional animations of the folding process of a characterized protein,
the program for the multi-dimensional animations of the characterized folding process of a protein and the meanwhile ensued post-translational in vitro modifications,
the program for simulating and predicting the folding process of a protein based on the characterized folding process of an homologous protein, and the de novo prediction of structural elements. The web-based software can be provided by Intra- or Internet-Servers.
the program bundle for the robot for characterization of protein folding.

These programs can be isolated depending on the application purposes, each as an independent product or can be offered in various bundles as part of the above mentioned assay kits and laboratory equipment.

### Embodiments

The present invention will now be illustrated by the following examples. These serve to illustrate certain preferred embodiments and aspects of the present invention, but are not be interpreted as the limiting scope thereof.

The embodiment relates to the characterization of the folding process of the overexpressed [alpha]-amylase inhibitor Parvulustat (Z-2685) from Streptomyces parvulus FH-1641 in Streptomyces lividans TK24. Parvulustat is due to its clearly defined pharmakophor structure, irreversible binding to the enzyme and low dissociation constant of 2.8×10⁻¹¹ M/L is a effective inhibitor of [alpha]-amylase, which reduces and slow down the uptake of glucose by the intestines into the blood and thus it is a potential antidiabetic agent for diabetes type II. Its three-dimensional structure has been elucidated by NMR analysis (Rehm et al, 2009; Pdb 2KER). The characterization of the folding process of Parvulustats for its biotechnological production and exploration of its pharmacokinetic properties has a major significance.

### Example 1

Analysis of the structural and physicochemical properties of the Parvulustats. Parvulustat (FIG. 1-A) consists of 78 amino acids with a molecular weight of 8282.09 Da. Its amino acid sequence is

ATGSPVAECVEYFQSWRYTDVHNGCADAVSVTVEYTHGQWAPCRVIEPGGWATFAGYGT
DGNYVTGLHTCDPATPSGV.

It has 4 cysteine residues, 5 prolines, 2 arginines, three tryptophans, 5 tyrosines, 2 phenylalanines, 3-sheets, 6-turns and two loop structures as a result of the two disulfide bridges, which each of them through thiols of cysteins 9-, -25 and -43, -70 are bound (FIG. 1C). It has 8 negative and 2 positively charged residues from 4 aspartic acids, 4 glutamic acids and 2 arginines. Its -amylase inhibitory activity centre in the triad Trp¹⁶ Arg¹⁷ Tyr¹⁸ as the pharmacophore is located at the -turn of the -sheet structure in the first loop structure. Its isoelectric point is 4.3. At pH 7.0 it is loaded from 5.9 net negative charges. The ratio between the hydrophilic and hydrophobic residues is 1:3. Its molecular surface is hydrophilic and polarized (FIG. 1-B). Parvulustat is a small compact protein that is completely denatured in 7M guanidine hydrochloride solution and that can after subsequent renaturation without loss of activity refold in the native state (FIG. 2-A).

According to the results of computer-assisted analysis, Parvulustat can have during the refolding 8 theoretically possible, by native and non-native disulfide formation resulting intermediates (FIG. 2-B), the fragment-mass detection pattern (FIG. 4-B) was predicted. It was also decided, that the separation of intermediates, the differentiation of their hydrodynamic sizes and determination of the order of these quantities is carried out simultaneously with native polyacrylamide gel electrophoresis and that the modification of the refolded Parvulustat is made with the charge neutral side-chain-specific reagent of Iodine acetamide on thiol groups of cysteine residues and that the subsequent proteolytic fragmentation of the separated intermediate in gel bands is carried out with trypsin in-gel digestion (FIG. 4-A).

The folding process of Parvulustat was characterized continuously on this basis according to the invention.

### Example 2

### Optimal Separation of the Unfolded Protein Sample with Maximized Hydrodynamic Sizes

Parvulustat was completely denatured, reduced and released of reducing agent.

Thereby the fraction of the optimal unfolded Parvulustat which has a maximum hydrodynamic size and its disulfide bonds were completely reduced to free thiol groups, were separated by liquid chromatography and prepared for re-oxidation and the dynamic modification in the next steps.

The denaturation and reduction of Parvulustat was done with the denaturation buffer containing 6M well known denaturant GdmCl (guanidinium chloride), 0.2 M Tris, 1 mM EDTA, pH 8.7 and reduction buffer of 0.2M well known reducing agent DTE (1,4-dithioerythritol), 6M GdmCl, 0.2 M Tris, 1 mM EDTA, pH 8.7. Parvulustat is easily aggregating in solution due to its highly polarized charges on the molecular surface. Its highest concentration during denaturation in 6M guanidine hydrochloride GdmCl solution should therefore not exceed 2.4 mg/ml in order to denature Parvulustat completely, to reduce and to prevent intermolecular disulfide formation. 5 mg Parvulustat in a 1.5 ml Eppendorf cap was first treated with 1.25 ml of denaturation buffer for 2 min and 5 min vortex and 15 min with ultrasound at 37° C. treated, then transferred to a 4 ml tubes in which above 1, 25 ml reduction buffer was transferred and after mixing and 5 minutes of ultrasonic treatment brought further for 1 hour at room temperature to complete denaturation and reduction of disulfide bridges.

The concentration of the Parvulustats in this solution is 2 mg/ml.

After denaturation and reduction the reduced Parvulustat must be separated thoroughly from the reducing agent DTE. Any trace of DTE in the Parvulustat solution would heavily affect the subsequent investigations. The remove of reducing agent DTE is performed with two serial gel filtration columns. Two PD-10 columns were first equilibrated each with 20 ml separation buffer at pH 2. Then, 2.5 ml reduced Parvulustat solution was transferred in the first column. After immersion, 2.5 ml separation buffer were added and at the same time, the eluted 2.5 ml Parvulustat solution is directly dropped into the second PD-10 column. Finally, 2.5 ml separation buffer was introduced into the second column while the Parvulustat solution was collected in 0.5 ml portions in 5×1, 5 ml tubes and then the concentration of the reduced Parvulustat and the number of reduced cysteine thiol groups (-SH) is determined.

The concentration of the reduced Parvulustat was used to control the content of the Parvulustat material in the solution and for calculation of the number of free thiol groups in the molecule. Here, the photometric measurements in the 280 nm UV range were used as the preferred method. This method uses the absorption of aromatic amino acids of 3 Tryptophane, 5 tyrosines and two disulfide bridges of Parvulustats. Its A₂₈₀ value at a concentration of 1 mg/ml is 2.92, which is much higher than that of most proteins generated values of 0.5 to 1.5. To maintain the linearity of the dependency of the absorption value of the concentration of Parvulustat the measured A₂₈₀ values should thereby not exceed 1. It is the concentration of the measured Parvulustat in the buffer solution in the range corresponding from 20 to 500 pg/ml, which is usually achieved by the appropriate dilution of the solution. During the measurements first Parvulustat samples were 10 fold diluted. Each 0.1 ml of reduced and from DTE removed Parvulustat solutions of 5 samples were transferred in 5×1, 5 ml tubes, which were previously, with 0.9 ml pH 7 phosphate buffer filled. After mixing, the absorbance values A₂₈₀ of the 5 solutions were measured in a UV spectrometer. The absorption coefficient ε₂₈₀ can be determined with the following first equation (Pace et al., 1995). The concentrations of Parvulustat were with the second equation below calculated directly without the use of the calibration method.

ε₂₈₀=(Trp)(5,500)+(Tyr)(1,490)+(disulfide bonds)(125)=(3)(5,500)+(5)(1,490)+(2)(125)=24200 (M⁻¹cm⁻¹)

C(mg/ml)=10·A₂₈₀/ε₂₈₀·d=A₂₈₀×82860 mg M⁻¹ cm⁻¹/24200 M⁻¹ cm⁻¹=3.42×A₂₈₀

It is essential for the successful characterization of the folding process, to fully denature and reduce the disulfide bonds of the examined protein to completely free thiol groups.

The determination of free thiol groups of 5 samples is used to get the best 5 samples with definitely fully reduced Parvulustat, that consist over all four free thiol groups (-SH) and thus has the maximum hydrodynamic size, as a starting material for the subsequent reoxidation, modification and interception of the intermediates. The determination of free thiol groups is carried out with Ellmansreagenz from 2 mg 5.5′-dithiobis (2-nitrobenzoic acid) (DTNB)/ml, 0.1 mM EDTA and 0.1 M phosphate buffer pH 7, 5. Each 0.1 ml of reduced Parvulustat solution of 5 samples was transferred in five 1.5 ml tubes, previously filled with 0.8 ml pH 7.5 phosphate buffer and mixed gently by tipping. Then in this each 5 tubes 0.1 ml Ellmans reagent was added. After mixing, the absorbance values of the 5 solutions at a wavelength of 412 nm in the spectrometer were measured. Here is Ellmans reagent (DTNB) by an SH-SS exchange reaction with reduced Parvulustat to the same stoichiometric product of TNB²⁻ with a bright yellow color and a molar absorption coefficient of 13600M⁻¹ cm⁻¹ at 412 nm brought.

The intensity of color is proportional to the free thiol groups in the Parvulustat molecule. The number of free thiol residues of Parvulustat in the sample solution is therefore by dividing the concentration of TNB²⁻ and Parvulustat in the solution stoichiometrically determined with the following equation (Riddles et al., 1983). It was spacified thereby, that that Parvulustat dispose in the first 0.5 ml eluate with a concentration of 1.2 mg/ml four fully reduced thiol groups and therefore the largest molecular hydrodynamic size (FIG. 2 C). The subsequent experiments were therefore performed with this sample.

$$\begin{matrix}number of the free \text{-} SH = Conc . of {TNB}^{2} / Conc . of Parvulustat \\ = \frac{( {A}_{TNB ( 412 nm )}^{- 2} / 13600 {M}^{- 1} {cm}^{- 1} )}{ ( {A}_{parvulustat} / 24200 {M}^{- 1} {cm}^{- 1} )}\end{matrix}$$

### Example 3

### Reoxidation and Dynamic Modification of the Reduced Parvulustat

The dynamic modification of the disulfide containing Parvulustat with 4 fully reduced thiol groups is carried out during its reoxidation. The reoxidation is used to form the native and possible non-native disulfide bridges of the Parvulustat from the reduced and free cystein thiol groups during its refolding. This bond formation between the thiols is not spontaneous, even if they are immediately adjacent. It depends on the particular redox potential, i.e. on the effective concentration of the appropiate electron donors and acceptors in the vicinity of thiols. For the formation of disulfide bonds, a suitable electron acceptor may be present.

For this there are numerous well-known oxidizing agents available. An effective and often used oxidant in the reoxidation experiments, glutathione in its reduced (GSH) and oxidized (GSSG) form (Creighton & Goldenberg, 1984), was used here with a preferred ratio of reduced (GSH) to oxidized (GSSG) glutathione 10:1.

The reoxidation was carried out by mixing the reoxidation buffer of 0.1M Tris/Ac, 10 mM EDTA, pH 8, the reoxidation solution of 10 mM GSH and 1 mM GSSG in reoxidation buffer and the fully reduced Parvulustat from the first eluate. 0.15 ml reoxidation solution is first mixed with 1, 16 ml reoxidation buffer in a 4 ml tube. The reoxidation is then started by addition of 0.19 ml of the first eluate and briefly vortex. The concentration of Parvulustat in this solution is 0.18 mg/ml. This 1.5 ml reoxidation mixture provides 15×100 μl samples for the dynamic modification, including intermediates of the trapped Parvulustat. The reoxidation is started when all samples for the subsequent modifications to trap the intermediates are carefully prepared.

The dynamic modification is carried out at various time intervals when a portion of the reoxidation mixture is separated and mixed with the well known side-chain-specific modification reagent iodine acetamide, which reacts irreversibly with all in the refolding and reoxidation remaining and accessible thiol groups through carboxamidomethylation and thereby converting cysteine residues to the neutral amido groups. First, each 20 μl modification agent of 0.6M iodine acetamide, 0.25M pH 7.5 are pipetted into 14 1.5 ml tubes, then the reoxidation as in the above-described in 1, 5 ml approach is started, subsequently in each case 100 μl reoxidation mixture after the planned time intervals of 1, 3, 6, 9, 15, 30, 45, 60, 90, 120, 150, 180, 210 and 240 minutes, altogether 14 times immediately were took out and transferred into tubes containing 20 μl modification agent, mixed, after 5 minutes incubation at room temperature, treated with liquid nitrogen and stored in the freezer (FIG. 3A). The concentration in these samples is Parvulustat respectively ca. 12 μl/100 μl. The refolded and reoxidated Parvulustat becomes modified depending on the accessibility of the thiol groups of cysteine residues in varying degrees to the structurally relatively stable intermediates, each with its own individual characteristics and is then ready for separation by gel electrophoresis and further identification.

### Example 4

### Separation and Quantification of the Intermediates and Two-Dimensional Representation of their Hydrodynamic Sizes as a Function of their Time of Formation

The separation of the modified intermediates and the simultaneous presentation of their hydrodynamic size with the inventively improved discontinuous native polyacrylamide gel electrophoresis in an apparatus from Hoefer Scientific SE600 after Davis and Ornstein.

In native gel electrophoresis, the migration of proteins is dependent on their net charges and conformations. Therefore, this type of electrophoresis is especially suitable for the separation and visualization of intermediates modified during protein refolding, which have an almost identical molecular weight but are structurally in different hydrodynamic sizes present. The gel should preferably be prepared one day before of use for complete polymerizing and kept in the refrigerator. The large gel (16 cm×18 cm×0, 1 cm) has 16 sample wells. In each case, a maximum of 120 μl sample containing 12-15 μg of protein is loaded.

In the first and last well of the gel no samples were applied because of the often occurring edge effect. In the second and third well of the gel in each case a 100 μl sample of native Parvulustats and a 100 μl sample of the fully reduced and with iodine acetamide modified Parvulustat after mixing with 20 μl sample buffer were applied. The 11 of 14 during the refolding and reoxdation by carboxamido methylation in the above mentioned time schedule of 1, 3, 6, 9, 15, 30, 45, 60, 120, 180 and 240 minutes modified Parvulustat samples are removed from the freezer and then concentrated in the SpeedVac up to 100 μl. Then 20 μl sample buffer were transferred in every 11 samples. After the mixture, 100 μl was applied in each case from these samples in the 11 wells of the gel. The gel (20% A, 3.25% T) runs for 2 hours at 120V for stacking gel and then for 4 hours at 180 V for resolving gel coupled with a cooling thermostat as well as the discontinuous replacement of the running buffer in cool room at 4° C. (FIG. 3 B).

After the gel run and Coomassie staining and destaining the intermediates trapped by modification during the refolding in the indicated time intervals were represented after their refolding and their simultaneously designed hydrodynamic size in the form of serial gel bands the 4 phase multi folding pathways model and accordingly two-dimensionally separated shown (FIG. 3 B).

On this gel image (FIG. 3-B) is to be noted, that total, during the refolding resulting intramolecular intermediates appear with different hydrodynamic sizes in 10 bands, that the types, number and quantity of the intermediates in the timing of the refolding is changing, that in the first phase of the super fast folding instinctively formed Seed structure of the folding pathways in the first gel band are represented, in the second phase occurred construction of the folding pathways or channels is reached within 15 to 30 minutes and thereby a refolding fingerprint profile is formed, that in the third phase along these paths or channels the folding takes more than 2 hours and that the further intramolecular rearrangement to the final completion of the native structure takes about 4 hours.

It can still be seen, that the completely denatured and reduced and right after the start of reoxidation with iodine acetamide modified Parvulustat, that due to the increased bulkiness of the 4 enlarged cysteine residues of the highest hydrodynamic molecular size and thus the slowest migration in the gel posses, whereat the native Parvulustat and renatured Parvulustat because of their compact structure at the same rate in gel moves. Furthermore, it is to see that the gel bands with identical heights from different time points of the refolding have identical intermediates and that the hydrodynamic size of the intermediates in gel band-10 is smaller than that of the native Parvulustats in gel band-9. The gel image was scanned and made available for the further identification of the intermediates in the gel bands.

### Example 5

### Fragmentation of Intermediates by in-Gel Digestion

The fragmentation of the intermediates from gel bands is performed with trypsin in-gel digestion. Trypsin (23.23 kDa) is one of the most commonly used serine proteases in protein analysis, especially for the production of peptide patterns. It catalyses the specific cleavage of the C-terminus of the peptide bond of arginine and lysine. Parvulustat has two arginine but no lysine. The first arginine is in the middle of the inhibitor activity center Trp16-Arg17-Arg44 Thr18 and the other is right next to the disulfide bridge Cys43-Cys70. The modified intermediates were cleaved by trypsin into 3 fragments, whereat two major fragments result from additional binding of the disulfide bonds between the fragments. Therefore, each intermediate with 5 fragments was described with exact mass (FIG. 4-A).

The trypsin digestion is made according to the invention by improved in-gel digestion of Sigma (trypsin proteomics grade, Product Code T6567). The 10 gel bands at the refolding time of 60 minutes were each carefully excised and subjected at once to the trypsin digestion with additional microwave and ultra-sonication treatment. Finally, all digestion mixtures were each transferred by pipette into Eppendorf tube and in the Speed Vac to 20 μl concentrated as a MALDI-MS sample. These 20 μl samples contained depending on the gel band 0.3-1, 5 μg of Parvulustat fragments, which have covered the requirements for MALDI-MS measurements completely.

Assuming that Parvulustat possess eight theoretically possible, by native and non-native disulfide occurring intermediates during the refolding (FIG. 2-B) and all these intermediates are each presented in 5 fragments with exact masses, the fragments detection pattern for all intermediates are produced in the form of a chart (FIG. 4-B) with the corresponding intermediates and their 5 fragments for comparison with measured mass spectral data and the helping tool.

### Example 6

### Mass Spectrometric Detection of Fragmented Intermediates

The mass detection of the proteolytic fragments of all intermediates from the gel bands is carried out using MALDI-MS measurements (FIG. 5). The intermediates of gel bands1 to -7 have relatively large hydrodynamic size and are all easy to digest proteolytically. The bonds between the cleaved fragments are due to the lack of support of the stable secondary structures relatively weak and therefore easy to solve with the MALDI measurement. This means that all these intermediates are present in 3 separate fragments in the MALDI spectrum. The absence of the second fragment of an intermediate in the gel band-8 indicates the existence of the intramolecular rearrangement of the disulfide bond, namely, that the non-native disulfide bridge Cys25-Cys43 to Cys25-Cys70 further rearranges to the native disulfide bond Cys43-Cys70. The suggestive trend for more than three fragments of a mixture of the intermediates in the gel bands-8 and -9 were confirmed by electro elution of the gel bands and subsequent chromatographic separation with the micro-columns.

The measured masses of all fragments, resulting from trypsin digestion were, according to their investigated 10 gel bands, in a table, in which the gradually decreasing hydrodynamic size of the corresponding gel band from -1 to -10 from top to bottom in the first column and their fragments in the rows from left to right in the order as fragments mass patterns recorded (FIG. 6). The intermediates based on the modification with their own fragment mass pattern were then characterized by comparison with the theoretical fragments detection pattern, differentiated and with a particular name in the adjoining cells, which are provided for the recognized intermediates located.

### Example 7

### Finding the Folding Pathway Identity of Intermediates

The folding pathway identity is crucial characteristic of an intermediate, with which the method according to invention differs as important characteristic from all well-known conventional methods. It can not alone be defined, but it can be identified to a certain folding pathways from its classified group membership.

The determination of the pathway identity of the intermediate starts with the group assignment of the identified intermediates. In the above described table, overall 12 stable intermediates from 10 gel bands were differentiated by their individual mass fragments pattern. There are 4 conformations of the reduced intermediate without disulfide bond formation in each case from gel band-2, -4, -6 and -7, labelled as CCCC-1, -2, -3 and -4, the 5 non-native conformations of the intermediates with the non-native disulfide bond Cys25-Cys43 each from gel band-3, -5, -8, -9, and -10, labelled as C25-C43-1, -2, -3, -4 and 5, a 1 briefly appearing non-native intermediate from gel band-8 with a non-native disulfide bond formation Cys25-Cys70, labelled as C25-C70, and 2 native intermediates Cys9-Cys25 and Cys43-Cys70 each from gel band-8 and 10, labelled as C9-C25 and C43-C70. The fully developed and modified Parvulustat in a gel band-1 was not considered to be intermediate.

All found intermediates can be classified according to each of their name and common structural context into 4 groups (FIG. 6). The 4 intermediate without disulfide bond with labelling CCCC-1, -2, -3 and -4 belong to the first group. The three non-native intermediates labelled as C25-C43-1, -2, and -3 belong to the second group. This also includes other non-native intermediate C25-C70, which is a product of the intramolecular rearrangement of intermediate C25-C43-4. The intermediate C25-C43-4 and 5 as well as the rearranged native intermediate C9-C25 form the third group. The most significant native intermediate C43-C70 represents the fourth group. For all 12 intermediates it is therefore possible to make conclusion about the folding pathways considering its decreasing hydrodynamic size and thereby identifying their own group identity, thus of the 4 groups to the 4 folding pathways. This way, the identity of all 12 intermediates was detected, each differentiating itself.

### Example 8

### Identification and Classification of the Intermediates

The identification of the 12 intermediates was performed by determining their hydrodynamic size, the time of formation during refolding and the determined folding pathways identity. Further, these 12 intermediates were classified into 4 groups and folding pathways accordingly to intermediates, depending to their roles being played for path-junction, -extension, -intersection, -crossover and -coincidence, they were classified.

The relative hydrodynamic sizes of the 12 intermediates are defined according to the different heights of the 10 gel bands. Their time of formation until 60 minutes after the start of refolding is crucial. Their folding pathway identity was found in the last section. Thus, all 12 intermediate, without quantifying their amount in the table (FIG. 6) are defined by their three characteristics.

From the illustrated gel image is to be determined (FIG. 7) that to the fourth category belonging intermediate referred to as C43-C70 was build 3 minutes after starting the refolding and therefore it belongs to the fastest folding pathway, that the intermediates of the first group labelled as CCCC 1, -2, -3 and -4, 6 minutes were build after the start of the refolding and therefore their belong to the fast folding pathway, that the intermediates of the second group labelled as C25-C43-1, -2, -3-4 were build 15 minutes after the start of the refolding and to the slow folding pathway belong, that the intermediate of the third group denoted as C9-C25 arises 30 minutes after the start of the refolding and up to permanently 3 hours appeared and therefore belongs to slowest folding pathway and that all state forms of folding pathways such as the coincidence, junction, extension, crossover, intersection to Molten globule state happens in gel band-8.

It was further found that the fastest folding pathway occurs only in the intermediate with native disulfide bridge Cys43-Cys70 and no intramolecular disulfide rearrangement appear, that in rapid folding grouped intermediates labelled as CCCC-1, -2, -3 and -4, the native conformations of the reduced Parvulustats are, which do not have a disulfide bridge, that in contrast the slow folding pathway, over the conformations of the intermediate with normative disulfide bridges Cys25-Cys43 runs and is accompanied by the intramolecular disulfide rearrangement of Cys25-Cys43 to Cys25-Cys70, and that the intramolecular disulfide rearrangement in the molten globule state with a hydrodynamic size analogous to the gel band-8 takes place.

### Example 9

### Characterization and Visualization of the Folding Process of Parvulustat in a 2- and 3-Dimensional Coordinate System

The characterization of the folding process of the Parvulustat occurs by parallel presentation of all, in the 4 folding pathways grouping 12 intermediates, first in a 2-dimensional coordinate system, wherein the gel bands corresponding to the hydrodynamic sizes of the 12 intermediates are plotted in each case against the time points of its first appearance up to 60 minutes (FIG. 8). In this connection, divided intermediates into 3 types were presented according to their hydrodynamic sizes and pathway identities, each in serial small symbolic graphs with gradually reduced size in varying shades of grey in this coordinate system, whereat the logical folding pathways were illustrated for demonstration with lines in shades of grey and strengths. In this coordinate system the y-ordinate with energy states/gel band of the intermediates against time of the folding sequence on the x-axis of ordinates was designated, because the energy states of the intermediates are proportional to their hydrodynamic sizes, corresponding to the different bands on the gel.

In this two-dimensional representation is presented clearly, that 12 intermediates divided into 3 types, namely the 4 conformations of the reduced Parvulustat without disulfide bridges, 2 intermediates with native disulfide bonds and 2 intermediates with non-native disulfide bridges in 6 conformations, formed during the refolding of Parvulustat, that these intermediates are folded into the 4 folding pathways as parallel events at different rates to the native Parvulustat, that the intermediate with first-formed native disulfide bond (Cys43-Cys70) as the basis of the folding pathway 1 dominates the procedure of folding, that through non-native disulfide bridge Cys25-Cys43 caused misfolding provoke the intramolecular disulfide rearrangements Cys25-Cys43 to Cys25-Cys70, continuing to Cys43-Cys70 and Cys25-Cys43 to Cys9-Cys25, which took hours and therefore slowed down the folding procedure, that in all this intramolecular disulfide rearrangements a compact molecular volume occure on to same structure levels of Molten globule state in band 8, that folding pathway-2 and -3 met at the intermediate with native Cys43-Cys70, the folding pathway-1 crossed and extended to renatured Parvulustat, that the folding pathway-4 from -way-3 branched off and leads to a disulfide rearrangement, that the intermediate with native disulfide bridge Cys9-Cys25 was the last occurring intermediate of refolding and that this last by intramolecular disulfide rearrangements resulting intermediate possess a hydrodynamic size smaller than native, i.e. it possess an energy state lower than the native Parvulustat. The folding procedure of the Parvulustats was completely characterized thereby.

The characterized folding process was also presented 3-dimensionally in a multi-coordinate system (FIG. 9), visualising each with the energy state according to the gel band and the hydrodynamic size as the y-ordinate, the time of formation as the x-ordinate and the folding pathway or -channel according to their folding pathways identity defined as z-ordinate, wherein the folding pathways of Parvulustat and its simplified processes was presented to 60^{th} minute of refolding.

### Example 10

### Product of the Process

The product of the inventive process regarding to Parvulustat includes et. al. the 12 dynamically modified, separated, and after their four characteristics identified intermediates and thus provides evidence that the cysteine-25 of the Parvulustat is involved in the formation of non-native disuifid bridge Cys25-Cys43 and is responsible for the misfolding of intermediates in 2 slow folding pathways, that these missfolding prevents the formation from the β-sheet structure resulted pharmacophore in the first loop of Parvulustat, slows down very the entire folding process and because of the abnormal activity and thereby initiated the intracellular degradation of these missfolded intermediates leads to a large loss in the in vivo biosynthesis of native Parvulustat.

### Example 11

### Use of the Product of the Process

The above as a product of the process described findings on the cause and consequence of misfolding of Pavulustats, were used here to improve its pharmacokinetic properties and increase its bio-productivity for specific modifications, redesigns and mergers of Parvulustat, where, for example, by the non-native disulfide bridge Cys25-Cys43 caused missfolding and the resulting abnormal effect is eliminated by targeted genetic exchange of the amino acid cysteine-25 of the Parvulustat for alanine and threonine.

### FIGURES

The following figures illustrate the invention.

FIG. 1 shows the NMR structure of the Parvulustat, its hydrophilic and polarized molecular surface of the front and back side, and his two loop structures resulting from two disulfide bridges.

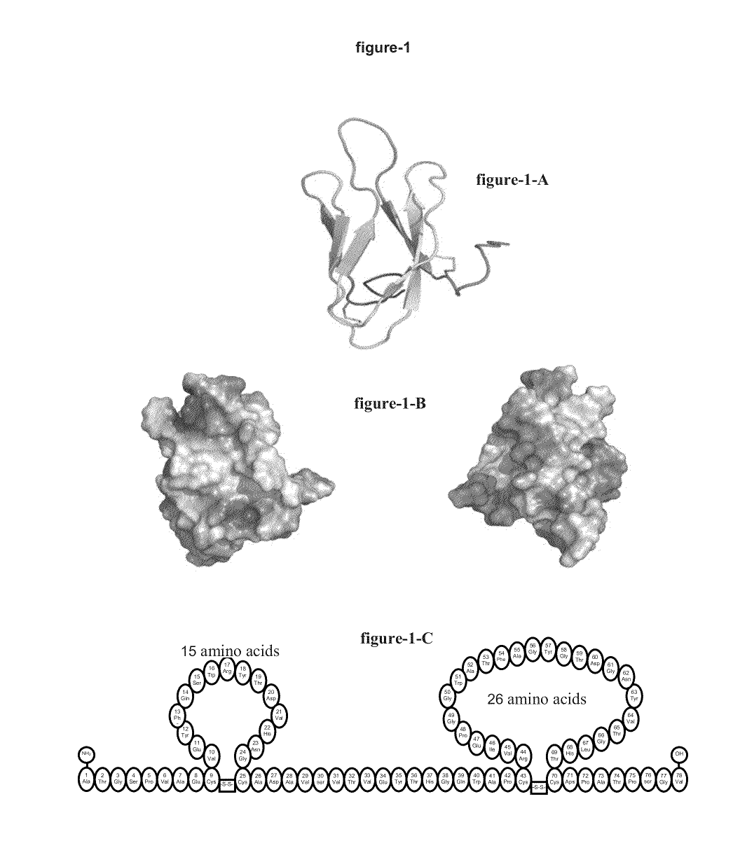

FIG. 2 shows the schematic task of characterizing Parvulustat in a 2-dimensional coordinate system, the 8 theoretically possible by native and non-native disulfide formation occurred intermediates in the refolding and for the modification selected side-chain-specific reagent of iodine acetatmide and the sample-collecting of the best optimally denaturated Parvulustat with maximum hydrodynamic sizes.

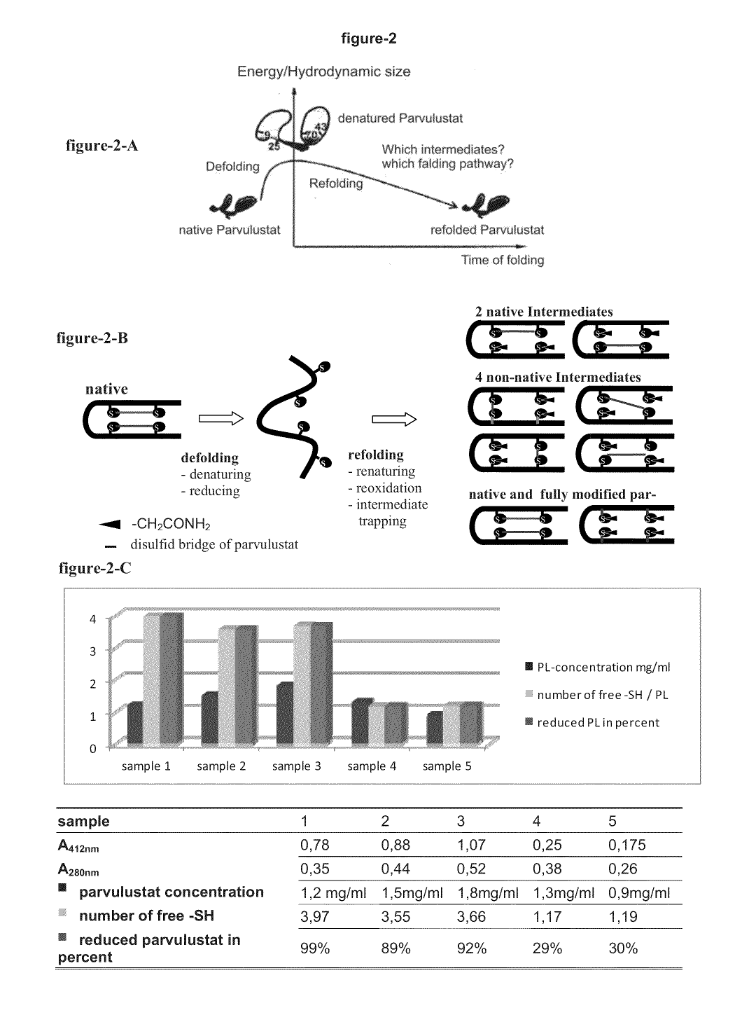

FIG. 3 presents the implementation of dynamic modification of the refolding Parvulustat at various time intervals and the 2-dimensional representation of the separated intermediates by native polyacrylamide gel with different hydrodynamic sizes and corresponding different gel bands as the y-ordinate, and the folding time in different time intervals as the x-ordinate.

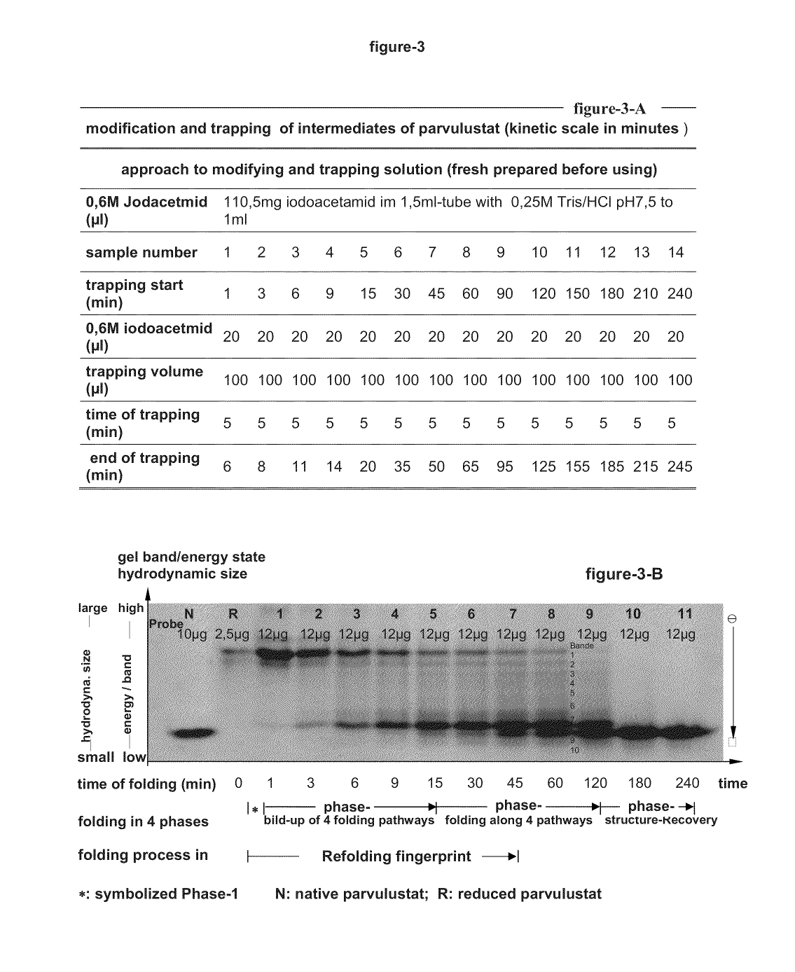

According to the invention defined 4 phases of folding and refolding the fingerprint profile are presented here.

FIG. 4 shows the trypsin cleavage pattern of the eight theoretically possible by native and non-native disulfide intermediates in the refolding and their fragments mass detection patterns in table form with announcements of the molecular masses of each fragment.

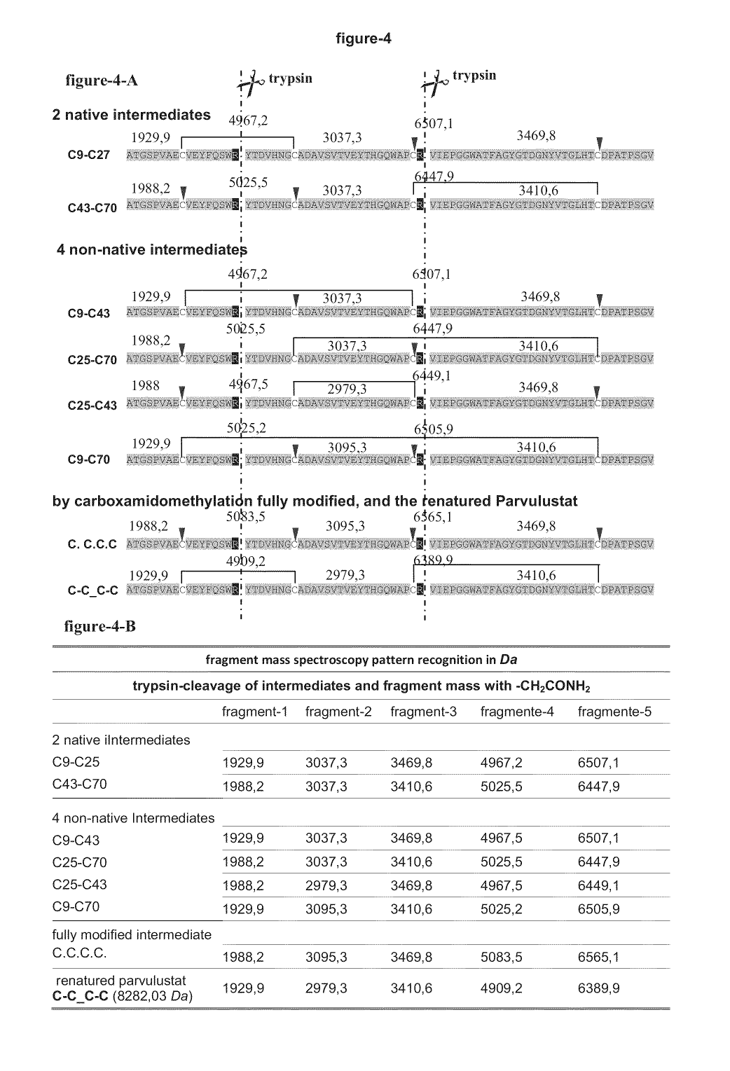

FIG. 5 shows the results of the mass detection of proteolytic fragments of all intermediates from 10 gel bands by MALDI-MS measurements.

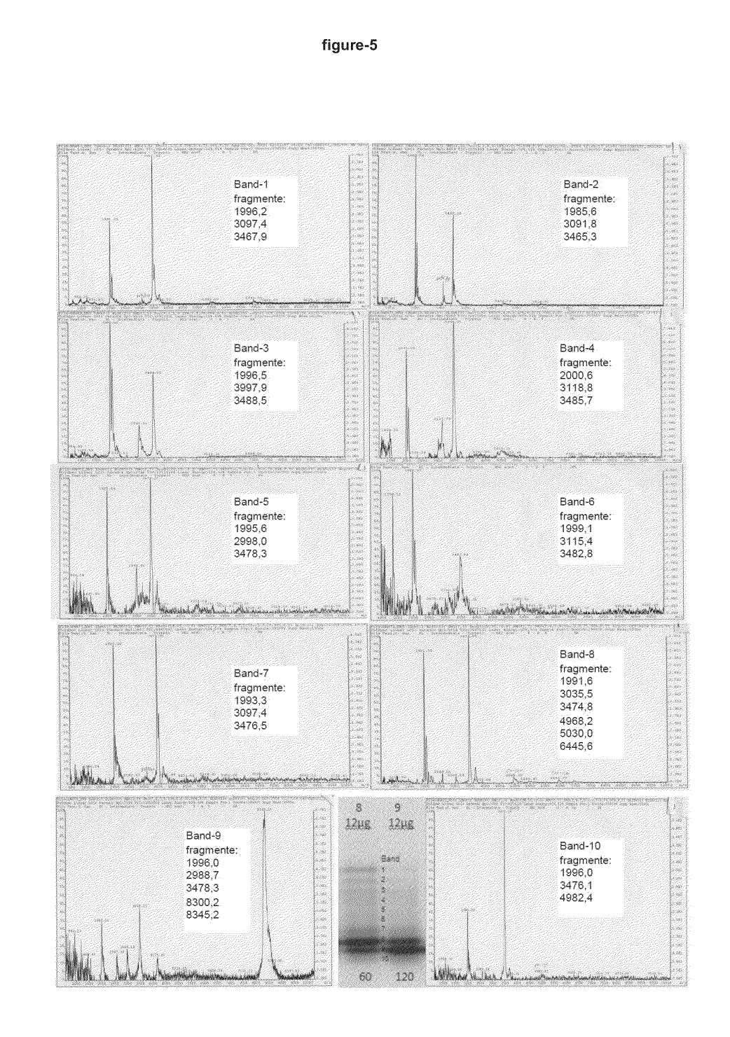

FIG. 6 gives a table of 12 differentiated and identified into 4 groups and 4 folding pathways associated intermediates from 10 gel bands by comparing the detected masses fragments of theoretical mass fragments pattern recognition.

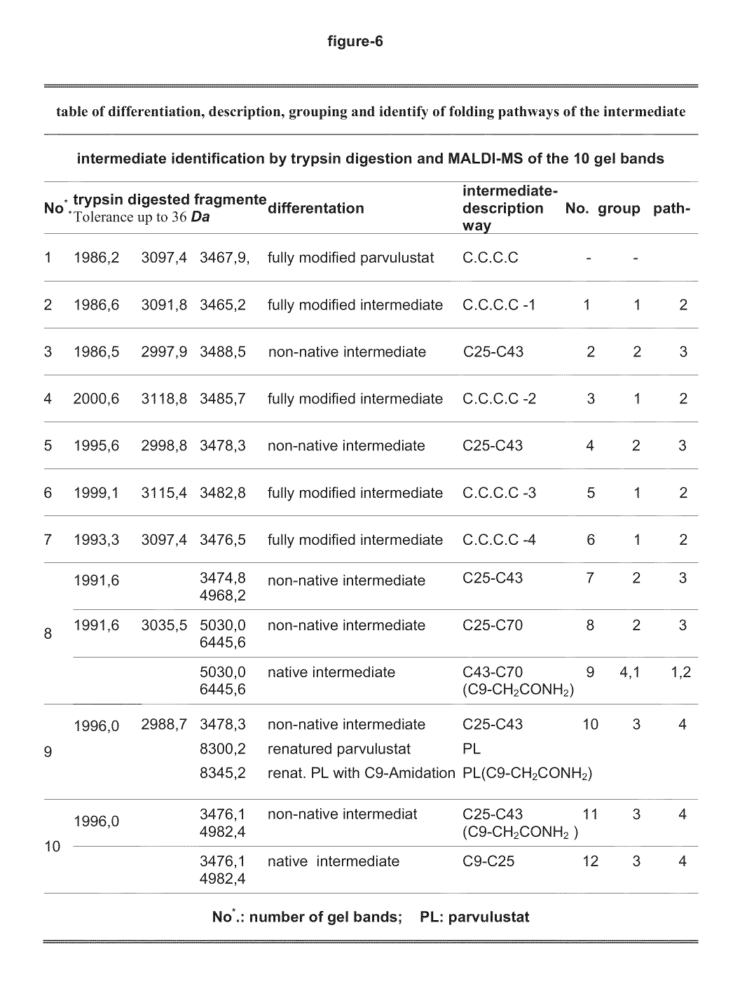

FIG. 7 shows the illustrated gel image corresponding 2-dimensional characterization of the illustrated folding process of Parvulustat.

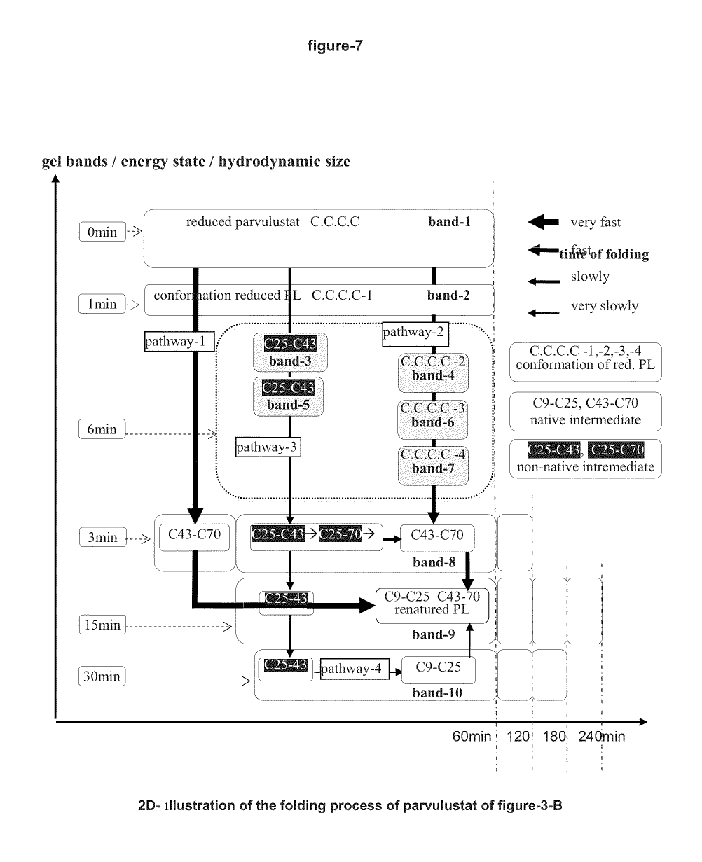

FIG. 8 shows the characterization and visualization of the folding process of Parvulustat up to the 60th minute in a 2-dimensional coordinate system.

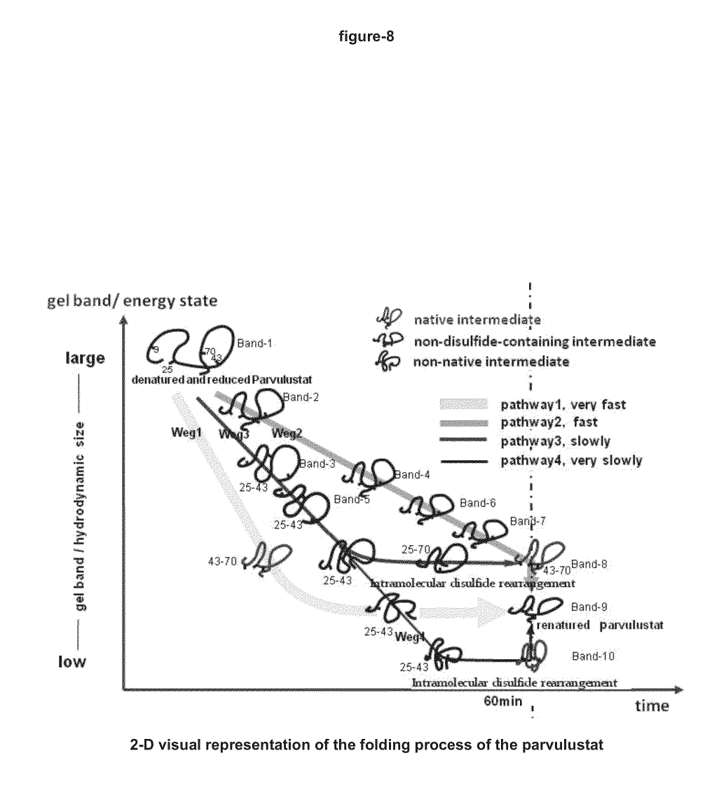

FIG. 9 shows the visualization of the folding pathways and their simplified processes of Parvulustat up to the 60th minute in a 3-dimensional coordinate system.

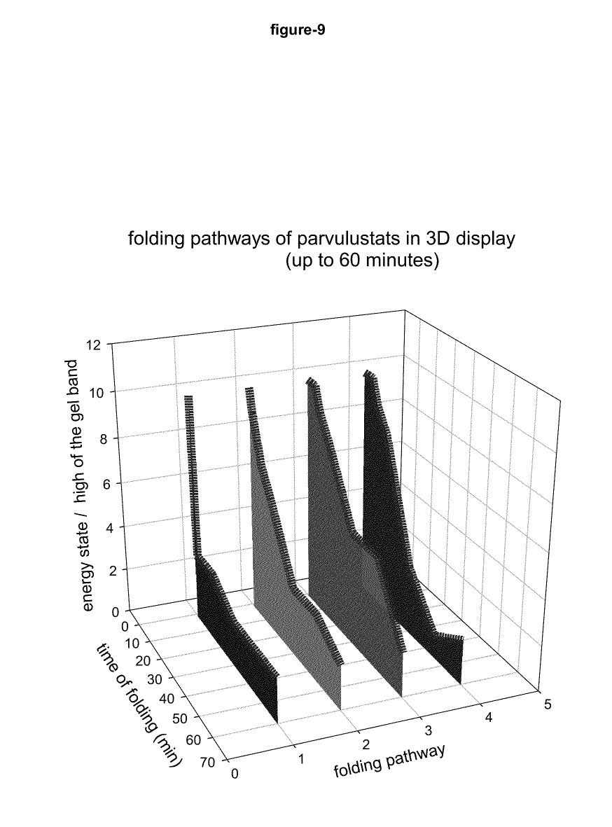

FIG. 10 shows the concept and the design of machines for the automated design process of the invention

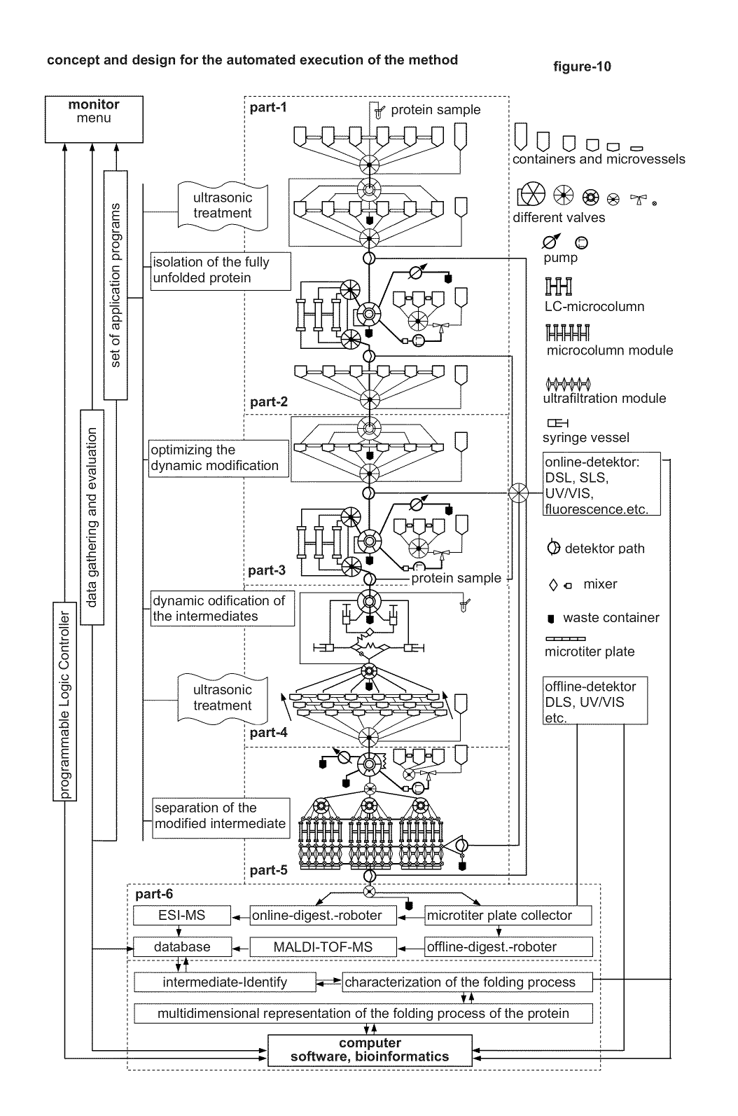

FIG. 11 shows a multi-dimensional energy landscape coordinate system assignable multi-folding pathway model. The model was illustrated with a graphical description, based on experiments and four folding phases and five functional zones summarized new findings of the course of protein folding.

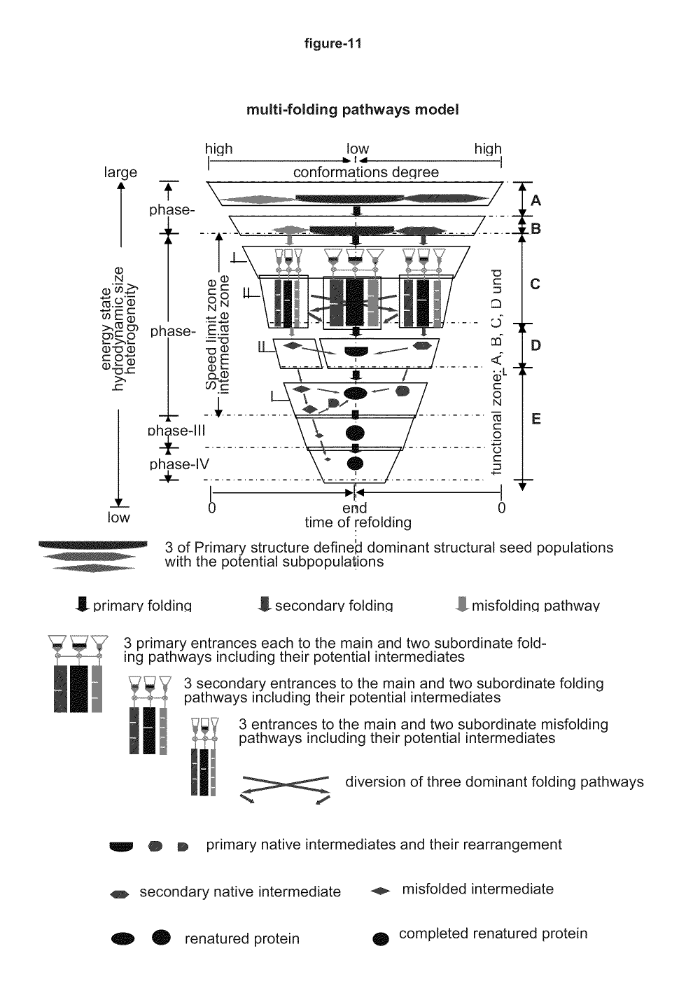

1. A method for the characterization and multidimensional visualization of the folding procedure of proteins, wherein all intermediates trapped by chemical modification at various time intervals during the refolding process are identified and digitized by at least 4 individual characteristics, namely its hydrodynamic size, the time of formation, the folding pathway identity, and the quantifiable amount, wherein a folding pathway consists of 4 phases, wherein the method is applicable for the characterization of the folding pathway of all proteins, and wherein the method comprises the following steps:
1) analysis of the protein to be characterized, on the basis of its primary, secondary and possibly three dimensional structure, its biological and physicochemical properties and its purpose, wherein a theoretical fragment mass identification pattern is created and the corresponding procedure and implementation are determined,
2) separation of the optimally unfolded fraction of the protein with maximized hydrodynamic size, wherein the protein is subjected to denaturing, if applicable reduction and removal of reducing agents, chromatographic separation and spectrometric identification,
3) dynamic modification of the protein intermediates during backfolding and if applicable reoxidation process, wherein portions of the refolding batch are separated at certain time intervals and the occurring intermediates are modified in different degree with appropriate reagents depending on the reactivity of the amino acids with according side chains reagents and are thereby converted to structurally relatively stable intermediates with its own individual characteristics, namely the hydrodynamic size, the time of formation, amount and the membership to a particular folding pathway,
4) separation and quantification of the intermediates and the depiction of their hydrodynamic sizes and, where applicable, their amount as a function of time of formation, where the intermediates, which were modified during the refolding process, are studied with electrophoresis or chromatography in conjunction with other spectroscopic methods, preferentially with dynamic light scattering (DLS) to analyze and quantify the individual characteristics of the intermediates, wherein a fingerprint profile of the refolding process is created, and the resulting modified intermediates are used for the next step,
5) fragmentation of the intermediates, wherein the modified intermediates are separated according to their hydrodynamic size and are proteolytically cleaved preferentially with Trypsin and optionally with the Endoproteases Lys-C, Glu-C and Asp-N and other Exoproteases or if necessary chemically in order to support differentiation of the fragments,
6) detection of the fragmented intermediates, wherein the molecular mass of all fragments and the larger fragments bound by disulfide bridges and/or crosslinkers are measured with mass spectrometry, and are documented in a database and classified in a fragment mass identification table for the purpose of identification, thereby MALDI-TOF-MS/MS (tandem mass spectroscopy) and/or MALDI-TOF_MS_PSD (post-source-delay) are used for the accurate differentiation of similar fragments,
7) determination of the intermediates folding pathway identity, where the type, the number and the position of the modifications serve as unique individual traits for every intermediate determined through the comparison of the mass spectrometrically documented number and mass of the fragments with the theoretical fragment mass identification pattern, and where the intermediates with the same traits are assigned into groups, and where the grouped intermediates point to further folding pathways, where this conclusion is compensated and complemented with criteria based on protein folding kinetics and protein evolution,
8) identification and classification of the intermediates, where every intermediate is identified by its individual characteristics, namely the hydrodynamic size, time of formation, folding pathway identity and the amount, and wherein these identified intermediates, which are sorted into groups are assigned to certain folding pathways, where these intermediates can meet three further criteria: namely the same modification characteristics, the reduction of their hydrodynamic size within their group and their course of formation and depletion over time within the distinct folding pathway,
9) characterization of the folding process with the help of the simultaneous multidimensional graphical depiction of all intermediates assigned to distinct folding pathways according to their identified characteristic, wherein the following characteristics of the folding process are determined: the dominant folding pathway to the native structure, the fastest and slowest folding pathway, pathways leading to misfolding, the folding kinetics in all pathways, the order of disulfide bond formation and/or crosslinking reaction, formation of knots and their impact on the protein folding process, intramolecular rearrangements, including disulfide rearrangements, folding pathway branching, extension, crossing and concurrence of intermediate forms, and
10) multidimensional visualization of the protein folding process, wherein the folding process is visualized and brought to animation using computer graphics for plotting the ordinates, defined by the four characteristics, in a multidimensional coordinate system, and wherein the diversity of visualization can be further extended by combination and transformation of these four characteristics.

2. The method according to claim 1, wherein step 3) is carried out in at least one of the following embodiments,
the dynamic modification of disulfide-containing proteins,
the dynamic modification of disulfide free proteins,
the dynamic modification of multi-domain proteins,
the in vitro simulated dynamic post and cotranslational modification,
the dynamic modification during in vitro simulated protein folding, and
the dynamic modification during in vitro protein biosynthesis, and with at least one of the following procedures,
the single modification, where residues of one kind of amino acid are marked with a single side-chain reagent under optimized reaction conditions,
the multiple modification, where the residues of more than one kind of amino acid react with one or more types of reagents by the use of different reaction conditions, or where the labeling is carried out in a single or in multiple batches first separately and then mixed, and
the internal internal crosslinker modification, where the internal double modification between two amino acids within one protein are carried out with bifunctional reagents under regulated reaction conditions in varied embodiments, and
with at least one of the following reaction kinetics,
timescale between nano and micro to milliseconds for very fast protein folding processes with a timescale of milliseconds to seconds for the formation of hydrogen bonds, the formation of secondary structural elements, and the hydrophobic collapse of the polypeptide chain,
timescale between microseconds to a minute for fast protein folding processes with a time scale of microseconds to several minutes for the fast folding phase and the formation of Intermediates belonging to separate folding pathways, and
timescale between milliseconds up to minutes for the slow protein folding with a timescale of minutes to hours or days for developing the intermediates, “molten globules” and further folding until the native state is reached, and
with at least one of the following chemicals,
denaturing agents of proteins,
reduction agents for disulfide containing proteins,
reoxidation agents including various components and compositions, specific for the modification of disulfide containing proteins,
side chain specific reagents without and/or with isotopic labeling,
side-chain-specific reagents with fluorescent or/and spin labeled reporter groups, e.g. reagents for DIGE (difference gel electrophoresis),
side-chain-specific zero length homo- and heterobifunctional reagents for the internal crosslinker marking, including reagents for photo affinity labeling,
biotinylation reagents,
reagents and/or enzymes for co- and posttranslational modifications, cell extracts,
foldases and/or chaperones,
candidate inhibitors of foldases and/or chaperones,
chemical and biological candidate agents for protein folding and degradation
chemical and biological candidate reagents for in vitro cotranslational modifications,
chemical and biological candidate reagents for in vitro posttranslational modifications,
chemical and biological candidate reagents for in vitro biosynthesis, and
auxiliary and stabilizing substances of the refolding of the protein.

3. The method according to claim 1, wherein step 4) is carried out manually, or automatically and in miniature, and with at least one of the following procedural steps,
accomplishment by electrophoresis, preferentially with the polyacrylamide gel electrophoresis focused on the global and hydrophilic proteins and with at least one of the following improvements,
connection of a buffer delivery system for the regulation of buffer pH, components, or strength during electrophoresis, using either an embedded permeable container, filled with the buffer solution and located in the buffer reservoir of the cathode, or a buffer manifold in the buffer reservoir of the cathode, connected by a thin tubing to an external container, in order to deliver the required buffer solution continuously with speed as needed, or in a discontinuous fashion in doses,
dynamic regulation and optimization of the separating resolution, where the dynamic differences of the charge- and polarity-distribution of the protein intermediates and their interactions with additional ion flow, are increased by adjustment of buffer-strength, -composition and -pH,
application of pulsed electrophoresis by using short duration increases in voltage, short duration changes in the polarity of the buffer components and/or their concentration, and short-duration reversals in polarity, combined with strong hydrophobic counterions in order to focus the intermediate gel bands, and enhance the distances between the intermediate gel bands,
application of variable gel-compositions and -forms, the suppression of diffusion of the intermediates caused by thermal effects, using Peltier cooling plates and a circulating liquid cooling system for heat drainage from the gel into ice containing cooling reservoir,
liquid chromatography, electrochromatography and field-flow fractionation coupled to spectrometric analytic devices preferentially dynamic light scattering (DLS) and static light scattering (SLS), for the separation of all protein intermediates and discrimination of their hydrodynamic size, mainly for the separation and size classification of the intermediates which are not suited for electrophoresis methods, such as proteins which are highly acidic, highly basic, hydrophobic, or which are membrane proteins and large proteins, on the micro-scale, analytical scale and semi-preparatory scale and with the following procedures
single column liquid chromatography with the application of micro gel filtration columns, filled with different separating stationary phases according to need or preference, or with the application of micro field-flow fractionation channels, in each case with coupling to spectrometric analysis for the direct determination of the order of the hydrodynamic sizes of the separated and individually collected intermediates,
multicolumn liquid chromatography coupled with spectrometric measurements, which are, according to need and wish, parallel or serial connected microcolumn combinations consisting of gelfiltration-, hydroxyapatit-, hydrophobic-, ion-exchange, reverse-phase- and affinity-chromatography, including the micro field-flow fractionation channel for the specific separation and size differentiation of the intermediates, especially those which have very similar hydrodynamic radii, but belong to the same or different folding pathways,
coupled spectrometric differentiation of hydrodynamic sizes of the separated and each in individual containers or in microtiter plates trapped intermediates and their quantification, preferably with DLS (Dynamic light scattering) and SLS (static light scattering), supplemented with fluorescence, UV, CD, NMR, EPR, and Fourier transformation,
specific differentiation of Intermediates in a single sample, which are either distributed in different hydrodynamic sizes or have very similar hydrodynamic sizes and may belong to different folding pathways, using adjusted DLS and SLS devices which include at least one of the following additional functions,
increased resolution of differentiation by extended measurement time,
Tm (melting point) differentiation by gradual temperature increase controlled by the program,
change in concentration of the samples in individual micro-vessels or in microtiter plates by dilution or concentration, which is accomplished by the built-in vacuum evaporation or ventilation,
change in the pH profile and buffer system by automated micro-titration or manual pipetting of the desired buffer composition,
determination and assessment of intrinsic viscosity and the zeta potential of the samples for further structural differentiation of the intermediates,
change the bio-rheologic properties of the samples by energy supply or discharge in the form of irradiation, heating and cooling, ultrasonic, microwaves, electric fields and magnetic fields,
analysis and classification of the differentiated intermediates by specially developed software, and
accomplishment with capillary electrophoresis preferably in coupling with DLS and SLS spectrometry, for the online automated and miniaturized separation of the intermediates and differentiation of their hydrodynamic sizes.

4. The method according to claim 1, wherein the folding process is characterized by determining the 3-dimensional structures of all or of the significant intermediates with an additional 5th characteristic, namely the structure of the intermediate, and is depicted in a multidimensional coordinate system with this integrated 5th ordinate and is visualized in many ways.

5. The method according to claim 1, wherein the method is used in investigation, examination and evaluation of changes in activity and functionality of a re-folding protein to its substrate, in different molecular environments, to improve or optimize the biotechnological production of a therapeutic protein.

6. The method according to claim 1, wherein the method is used in protein engineering, and where findings about folding processes of the underlying protein engineering and the resulting changes in functionality and activity of a protein are verified, examined and evaluated.

7. The method according to claim 1, wherein the method is used in the dynamic characterization and quantification of the processes of the in vitro simulated biosynthesis and possibly occurring co- and posttranslational modifications for the condition optimization of the biotechnological production of in vitro post-translationally modified protein therapeutics and scanning of the chemical and biological auxiliary or inhibitory substances to the co- and/or post-translational modifications, whereas the characterization of the processes of the in vitro simulated dynamic co- and post-translational modifications are carried out in a defined manner and are subjected to appropriate comparisons and evaluations.

8. The method according to claim 1, wherein the method is used in the dynamic characterization of the refolding process of proteins, investigated by in vitro simulation, during their in vivo post-translational modification for process development of their biotechnological production, where the characterization of these modifications follows the defined steps and the technology of protein immobilization and protein chip fabrication is used.

9. The method according to claim 1, wherein the method is used in the search for biological or chemical substances to influence the protein folding process, wherein the selected biological and/or chemical candidate inhibitors and/or auxiliary substances are included in the characterization procedure of the refolding process of to the protein.

10. The method according to claim 1, wherein the method is used in the investigation of the effect of foldases or chaperones on protein folding, wherein the effect is defined by comparison of the characterized refolding experiments with and without the foldases or chaperones to be examined.

11. The method according to claim 1, wherein the method is used in the search for biological and chemical inhibitors of foldases, chaperones and agents of protein degradation, where the protein to be examined is subjected to characterizations, comparisons and evaluations of the folding process in parallel experiments for each given foldase, chaperone and agent of protein degradation first without and then in the presence of the respective candidate inhibitors.

12. The method according to claim 1, wherein the method is used in the investigation of controlled self-assembly and polymerization of the polypeptides or proteins during their refolding process, for the development and production of nano-protein materials, wherein the initial events of self-assembly and polymerization in presence of biological and/or chemical factors in varying physiological and biochemical conditions are characterized and evaluated.

13. The method according to claim 1, wherein the method is used in the search for pharmacological chaperones against diseases caused by proteins (proteopathies), wherein the effect of certain biological and/or chemical stabilizing substances which specifically bind to unfolded proteins and improve the folding and stabilize the protein structure, or which mask the hydrophobic domains of misfolded proteins and increase the solubility and prevent aggregation of unfolded proteins, is detected by characterization of the refolding of proteopathic proteins.

14. The method according to claim 1, wherein the method is used in the characterization of first the process of the refolding of PrP^{C} (Prion protein cellular) to PrP^{SC} (prion protein Scrapie; pathogenic form of the prion protein) including occurring aggregation, and secondly the reversal of the PrP^{SC} to PrP^{C} including the depletion of aggregation in order to clarify the pathogenesis or to search for treatment options and possibilities for prevention, wherein the folding processes of a prion protein is characterized in parallel experiments with biological and/or chemical substances influencing the folding process under destabilizing conditions, and is compared with its previously characterized folding process without influencing substances.

15. The method according to claim 1, wherein the method is used in the dynamic characterization of the folding process of a protein to be examined for the study of protein aging due to isomerization, deamidation and racemization protein degradation caused by free-radical action, oxidative stress and environmental influences, where the protein to be examined is subjected during its refolding process to either a catalytically accelerated isomerization, deamidation and racemization or supply of photochemical and thermal energy, or a radical-initiated action, oxidative stress and environmental factors which cause modification, and its thus altered refolding process is characterized and compared with the previously characterized process without influencing factors.

16. The method according to claim 1, wherein the method is used in the classification (taxonomy) of the proteins and investigation of protein evolution on a new level of protein folding, where the characterized folding process, as an individual fingerprint of each protein, provides the functional and evolutionary relationships of proteins and is integrated as an additional criterion in protein classification which so far is only determined by the structure, topology, homology and evolutionary relationship of the proteins.

17. The method according to claim 1, wherein the method is used in the dynamic characterization of the folding process of complex formation of nucleotide-, glyko- and lipo-proteins to investigate the formation process and thus accompanied changes in activity and functionality and to find chemical and biological agents acting on the complex formation, whereas the refolding process is first characterized only for the protein and its complex and then in time intervals during the refolding process the protein is brought together with the complex forming substances and the characterizations of these approaches with and without supply of the substrate of this protein complex under varying chemical and biological factors are analyzed and compared.

18. The method according to claim 1, wherein the method is used in the diagnosis and prognosis of diseases caused by protein misfolding, where the changes in the folding process of the disease-related proteins are characterized and defined as criteria for diagnosis and prognosis of certain diseases.

19-22. (canceled)

23. A means for performing the method according to claim 1, wherein said means is one or more selected from the group consisting of kits, equipment, devices, software, and machines, which are needed for manual or automated execution of at least one step of the method.

24-27. (canceled)

28. A database containing descriptions of protein folding processes, which descriptions have been determined by carrying out the method according to claim 1.

29. (canceled)


SEQUENCE LISTING (1 sequence)

SEQ ID NO: 1 - protein, 78 aa, Streptomyces parvulus
ATGSPVAECVEYFQSWRYTDVHNGCADAVSVTVEYTHGQWAPCRVIEPGGWATFAGYGTDGNYVTGLHTCDPATPSGV